(19) United States
(12) Patent Application Publication
(10) Pub. No.: US 2025/0221686 A1
(43) Pub. Date: Jul. 10, 2025

(54) IMAGE DIAGNOSIS SYSTEM, IMAGE DIAGNOSIS METHOD, AND STORAGE MEDIUM

(71) Applicant: TERUMO KABUSHIKI KAISHA, Tokyo (JP)

(72) Inventor: Kohtaroh KUSU, Ebina Kanagawa (JP)

(21) Appl. No.: 19/094,734

(22) Filed: Mar. 28, 2025

Related U.S. Application Data
(63) Continuation of application No. PCT/JP2023/035479, filed on Sep. 28, 2023.

(30) Foreign Application Priority Data
Sep. 30, 2022 (JP) 2022-158098

(51) Int. Cl. A61B 8/12; A61B 8/00; A61B 8/08; A61B 8/14; A61B 90/00; G06T 7/62

(52) U.S. Cl. CPC A61B 8/12; A61B 8/0891; A61B 8/14; A61B 8/4416; A61B 8/463; A61B 8/5223; A61B 90/39; G06T 7/62; A61B 2090/3966; A61B 2560/0462; G06T 2207/10084; G06T 2207/10101; G06T 2207/10132; G06T 2207/30096; G06T 2207/30101

(57) ABSTRACT

A system includes a catheter insertable into a blood vessel and including: a first sensor configured to transmit ultrasonic waves and receive the waves reflected by the vessel, and a second sensor configured to emit light and receive the light reflected by the vessel, and a processor configured to perform the steps of: generating an ultrasonic tomographic image of the vessel based on the waves and an optical coherence tomographic image of the vessel based on the light, specifying a location of a lesion in the vessel based on the images, generating first feature data related to the lesion from the ultrasonic image and second feature data related to the lesion from the optical image, inputting the feature data into a model to generate risk information related to an onset risk of ischemic heart disease, and outputting the risk information.

### CROSS-REFERENCE TO RELATED APPLICATION

This application is a continuation of International Patent Application No. PCT/JP2023/035479 filed Sep. 28, 2023, which is based upon and claims the benefit of priority from Japanese Patent Application No. 2022-158098, filed Sep. 30, 2022, the entire contents of which are incorporated herein by reference.

### BACKGROUND

### Technical Field

Embodiments described herein relate to an image diagnosis system, an image diagnosis method, and a storage medium.

### Related Art

A medical image of a blood vessel such as an ultrasonic tomographic image is generated by an intravascular ultrasound (IVUS) method using a catheter for performing an ultrasonic inspection of the blood vessel. Meanwhile, for the purpose of assisting a doctor in making a diagnosis, a technology of adding information to a medical image by image processing or machine learning has been developed. For example, features of objects such as a luminal wall and a stent can be identified in a blood vessel image using such technology.

### SUMMARY

However, with the conventional technique, it is difficult to predict an onset risk of ischemic heart disease.

Embodiments provide an image diagnosis system and an image diagnosis method capable of predicting and outputting an onset risk of ischemic heart disease.

In one embodiment, an image diagnosis system comprises a catheter insertable into a blood vessel and including: a first sensor configured to transmit ultrasonic waves and receive the waves reflected by the blood vessel while the catheter is inserted in the blood vessel, and a second sensor configured to emit light and receive the light reflected by the blood vessel while the catheter is inserted in the blood vessel; a memory; and a processor configured to execute a program that is stored in the memory to perform the steps of: generating an ultrasonic tomographic image of the blood vessel based on the reflected waves received by the first sensor and an optical coherence tomographic image of the blood vessel based on the reflected light received by the second sensor, specifying a location of a lesion in the blood vessel based on the ultrasonic tomographic image and the optical coherence tomographic image, generating first feature data related to the lesion from the ultrasonic tomographic image and second feature data related to the lesion from the optical coherence tomographic image, inputting the first and second feature data into a computer model to generate risk information related to an onset risk of ischemic heart disease, the computer model having been trained with feature data of different lesions and a plurality of answer information corresponding to the different lesions, each answer information indicating whether the ischemic heart disease has developed from a corresponding one of the lesions, and outputting the risk information related to the onset risk of ischemic heart disease.

In one aspect, an onset risk of ischemic heart disease can be predicted and output.

### BRIEF DESCRIPTION OF DRAWINGS

FIG. 1 is a schematic diagram illustrating an image diagnosis apparatus in a first embodiment.

FIG. 2 is a schematic diagram illustrating an image diagnosis catheter.

FIG. 3 is a diagram illustrating a cross section of a blood vessel through which a sensor unit is inserted.

FIG. 4A is a diagram for explaining a tomographic image.

FIG. 4B is a diagram for explaining a tomographic image.

FIG. 5 is a block diagram illustrating an image processing apparatus.

FIG. 6 is a diagram for explaining a process executed by the image processing apparatus.

FIG. 7 is a schematic diagram illustrating a computer learning model in the first embodiment.

FIG. 8 is a flowchart for explaining a process executed by the image processing apparatus in the first embodiment.

FIG. 9 is a schematic diagram illustrating an output example of an onset risk.

FIG. 10 is a schematic diagram illustrating an output example of an onset risk.

FIG. 11 is a schematic diagram illustrating a computer learning model in a second embodiment.

FIG. 12 is a diagram for explaining a process executed in a third embodiment.

FIG. 13 is a schematic diagram illustrating a computer learning model in the third embodiment.

FIG. 14 is a flowchart for explaining a process executed by an image processing apparatus in the third embodiment.

FIG. 15 is a schematic diagram illustrating a computer learning model in a fourth embodiment.

FIG. 16 is a schematic diagram illustrating a computer learning model in a fifth embodiment.

FIG. 17 is a schematic diagram illustrating a computer learning model in a sixth embodiment.

FIG. 18 is a diagram for explaining a process in a seventh embodiment.

FIG. 19 is a schematic diagram illustrating a learning model in the seventh embodiment.

FIG. 20 is a flowchart for explaining a process executed by an image processing apparatus in the seventh embodiment.

FIG. 21 is a schematic diagram illustrating a computer learning model in an eighth embodiment.

FIG. 22 is a schematic diagram illustrating a computer learning model in a ninth embodiment.

### DETAILED DESCRIPTION

Hereinafter, embodiments of the present invention will be described in detail with reference to the drawings illustrating embodiments thereof.

### First Embodiment

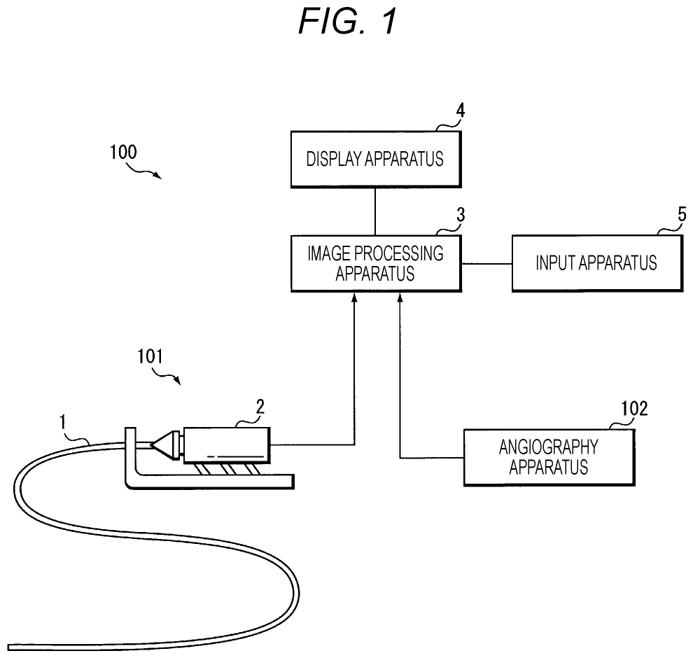

FIG. 1 is a schematic diagram illustrating an image diagnosis system 100 according to a first embodiment. In the present embodiment, an image diagnosis apparatus using a dual type catheter having functions of both intravascular ultrasound diagnosis method (IVUS) and optical coherence tomography (OCT) will be described. In the dual type catheter, a mode of acquiring an ultrasonic tomographic image only by IVUS, a mode of acquiring an optical coherence tomographic image only by OCT, and a mode of acquiring both tomographic images by IVUS and OCT are provided, and these modes can be switched and used. Hereinafter, the ultrasonic tomographic image and the optical coherence tomographic image are also referred to as an IVUS image and an OCT image, respectively. In a case where it is not necessary to distinguish and describe the IVUS image and the OCT image, they are also simply described as tomographic images.

The image diagnosis system 100 includes an intravascular inspection apparatus 101, an angiography apparatus 102, an image processing apparatus 3, a display apparatus 4, and an input apparatus 5. The intravascular inspection apparatus 101 includes an image diagnosis catheter 1 and a motor drive unit (MDU) 2. The image diagnosis catheter 1 is connected to the image processing apparatus 3 via the MDU 2. The display apparatus 4 and the input apparatus 5 are connected to the image processing apparatus 3. The display apparatus 4 is, for example, a liquid crystal display, an organic EL display, or the like, and the input apparatus 5 is, for example, a keyboard, a mouse, a touch panel, a microphone, or the like. The input apparatus 5 and the image processing apparatus 3 may be integrated into one apparatus. Furthermore, the input apparatus 5 may be a sensor that receives a gesture input, a line-of-sight input, or the like.

The angiography apparatus 102 is connected to the image processing apparatus 3. The angiography apparatus 102 is an angiography apparatus that images a blood vessel from outside a living body of a patient using X-rays while injecting a contrast agent into the blood vessel of the patient to obtain an angiographic image that is a fluoroscopic image of the blood vessel. The angiography apparatus 102 includes an X-ray source and an X-ray sensor, and images an X-ray fluoroscopic image of the patient by the X-ray sensor receiving X-rays emitted from the X-ray source. Note that the image diagnosis catheter 1 has a marker that does not transmit X-rays, and the position of the image diagnosis catheter 1 (i.e., the marker) is visualized in the angiographic image. The angiography apparatus 102 outputs the angiographic image obtained by imaging to the image processing apparatus 3, and causes the display apparatus 4 to display the angiographic image via the image processing apparatus 3. Note that the display apparatus 4 displays the angiographic image and the tomographic image imaged using the image diagnosis catheter 1.

Note that, in the present embodiment, the image processing apparatus 3 is connected to the angiography apparatus 102 that images two-dimensional angiographic images. However, the present invention is not limited to the angiography apparatus 102 as long as it is an apparatus that images a luminal organ of a patient and the image diagnosis catheter 1 from a plurality of directions outside the living body.

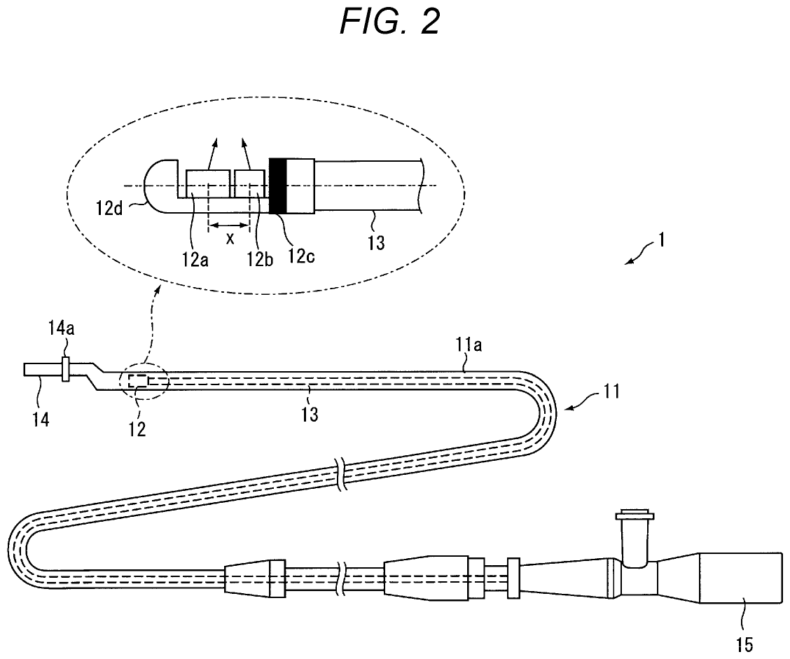

FIG. 2 is a schematic diagram illustrating the image diagnosis catheter 1. Note that a region indicated by a one-dot chain line on an upper side in FIG. 2 is an enlarged view of a region indicated by a one-dot chain line on a lower side. The image diagnosis catheter 1 includes a probe 11 and a connector portion 15 disposed at an end of the probe 11. The probe 11 is connected to the MDU 2 via the connector portion 15. In the following description, a side far from the connector portion 15 of the image diagnosis catheter 1 will be referred to as a distal end side, and a side of the connector portion 15 will be referred to as a proximal end side. The probe 11 includes a catheter sheath 11a, and a guide wire insertion portion 14 through which a guide wire can be inserted is provided at a distal portion thereof. The guide wire insertion portion 14 is a guide wire lumen that receives a guide wire previously inserted into a blood vessel and guides the probe 11 to an affected part by the guide wire. The catheter sheath 11a forms a tube portion continuous from a connection portion with the guide wire insertion portion 14 to a connection portion with the connector portion 15. A shaft 13 is inserted into the catheter sheath 11a, and a sensor unit 12 is connected to a distal end side of the shaft 13.

The sensor unit 12 includes a housing 12d, and a distal end side of the housing 12d is formed in a hemispherical shape in order to suppress friction and catching with an inner surface of the catheter sheath 11a. In the housing 12d, an ultrasound transmitter and receiver 12a (hereinafter referred to as an IVUS sensor 12a) that transmits ultrasonic waves into a blood vessel and receives reflected waves from the blood vessel and an optical transmitter and receiver 12b (hereinafter referred to as an OCT sensor 12b) that transmits near-infrared light into the blood vessel and receives reflected light from the inside of the blood vessel are disposed. In the example illustrated in FIG. 2, the IVUS sensor 12a is provided on the distal end side of the probe 11, the OCT sensor 12b is provided on the proximal end side thereof, and the IVUS sensor 12a and the OCT sensor 12b are arranged apart from each other by a distance x along the axial direction on the central axis (i.e., the two-dot chain line in FIG. 2) of the shaft 13. In the image diagnosis catheter 1, the IVUS sensor 12a and the OCT sensor 12b are attached such that a radial direction of the shaft 13 that is approximately 90 degrees with respect to the axial direction of the shaft 13 is set as a transmission/reception direction of an ultrasonic wave or near-infrared light. Note that the IVUS sensor 12a and the OCT sensor 12b are desirably attached slightly shifted from the radial direction so as not to receive a reflected wave or reflected light on the inner surface of the catheter sheath 11a. In the present embodiment, for example, as indicated by an arrow in FIG. 2, the irradiation direction of the ultrasonic wave from the IVUS sensor 12a is inclined to the proximal end side with respect to the radial direction of the shaft 13, and the irradiation direction of the near-infrared light from the OCT sensor 12b is inclined to the distal end side with respect to the radial direction of the shaft 13.

An electric signal cable (not illustrated) connected to the IVUS sensor 12a and an optical fiber cable (not illustrated) connected to the OCT sensor 12b are inserted into the shaft 13. The probe 11 is inserted into the blood vessel from the distal end side. The sensor unit 12 and the shaft 13 can move forward or rearward inside the catheter sheath 11a and can rotate in a circumferential direction. The sensor unit 12 and the shaft 13 rotate about the central axis of the shaft 13 as a rotation axis. In the image diagnosis system 100, by using an imaging core including the sensor unit 12 and the shaft 13, a state of the blood vessel therein is measured by an ultrasonic tomographic image (i.e., an IVUS image) captured from the inside of the blood vessel or an optical coherence tomographic image (i.e., an OCT image) captured from the inside of the blood vessel.

The MDU 2 is a drive apparatus to which the probe 11 (i.e., the image diagnosis catheter 1) is detachably attached by the connector portion 15, and controls the operation of the image diagnosis catheter 1 inserted into the blood vessel by driving a built-in motor according to an operation of a medical worker. For example, the MDU 2 performs a pull-back operation of rotating the sensor unit 12 and the shaft 13 inserted into the probe 11 in the circumferential direction while pulling the sensor unit 12 and the shaft 13 toward the MDU 2 side at a constant speed. The sensor unit 12 continuously scans the inside of the blood vessel at predetermined time intervals while moving and rotating from the distal end side to the proximal end side by the pull-back operation and continuously captures a plurality of transverse tomographic images substantially perpendicular to the probe 11 at predetermined intervals. The MDU 2 outputs reflected wave data of an ultrasonic wave received by the IVUS sensor 12a and reflected light data received by the OCT sensor 12b to the image processing apparatus 3.

The image processing apparatus 3 acquires a signal data set which is the reflected wave data of the ultrasonic wave received by the IVUS sensor 12a and a signal data set which is reflected light data received by the OCT sensor 12b via the MDU 2. The image processing apparatus 3 generates ultrasonic line data from a signal data set of the ultrasonic waves, and generates an ultrasonic tomographic image (i.e., an IVUS image) obtained by imaging a transverse section of the blood vessel based on the generated ultrasonic line data. In addition, the image processing apparatus 3 generates optical line data from the signal data set of the reflected light, and generates an optical coherence tomographic image (i.e., an OCT image) obtained by imaging a transverse section of the blood vessel based on the generated optical line data. Here, the signal data set acquired by the IVUS sensor 12a and the OCT sensor 12b and the tomographic image generated from the signal data set will be described.

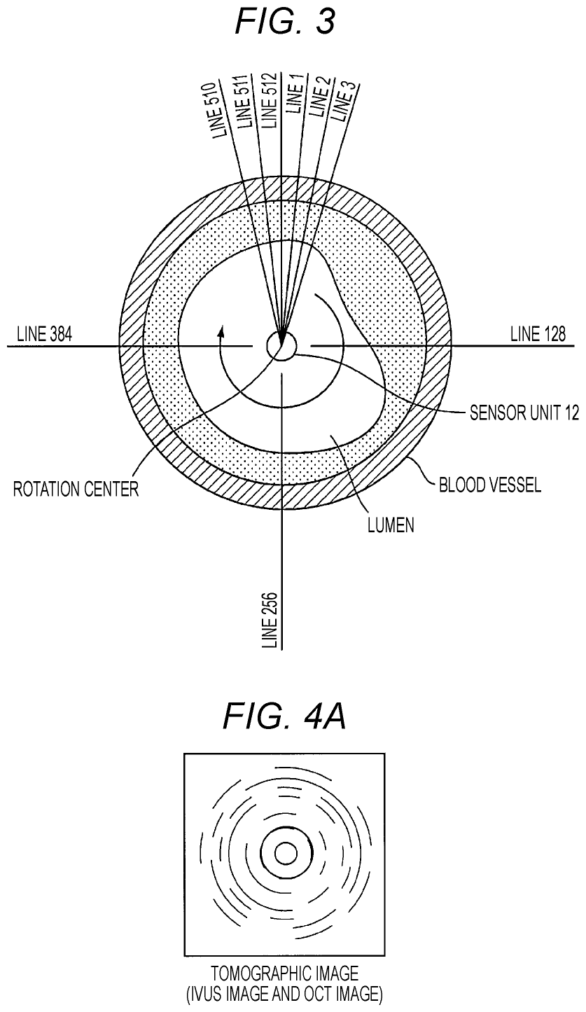

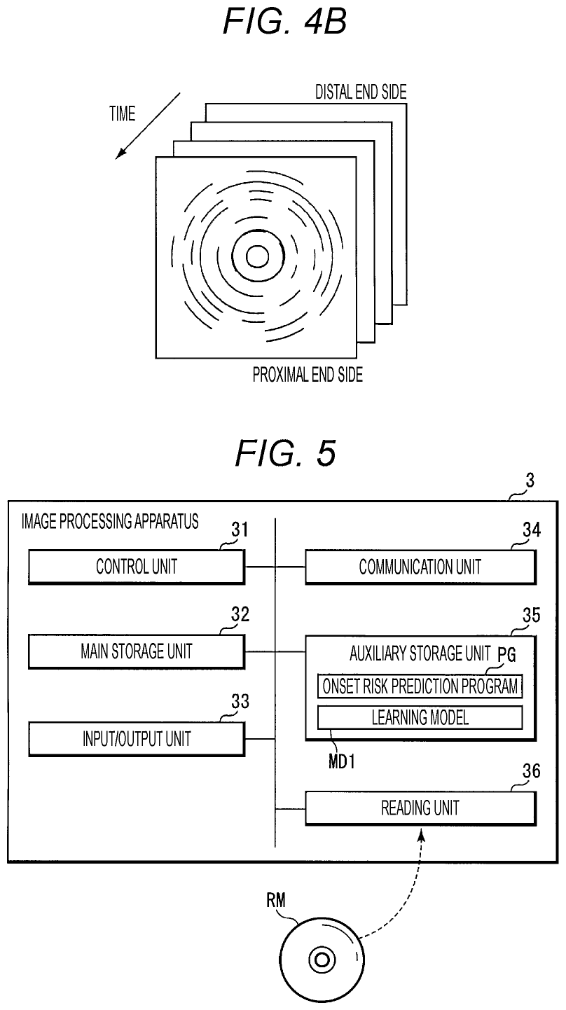

FIG. 3 is a diagram illustrating a cross section of a blood vessel through which the sensor unit 12 is inserted, and FIGS. 4A and 4B are diagrams illustrating tomographic images. First, with reference to FIG. 3, operations of the IVUS sensor 12a and the OCT sensor 12b in the blood vessel, and signal data sets (i.e., ultrasonic line data and optical line data) acquired by the IVUS sensor 12a and the OCT sensor 12b will be described. When the imaging of the tomographic image is started in a state where the imaging core is inserted into the blood vessel, the imaging core rotates about a central axis of the shaft 13 as a rotation center in a direction indicated by an arrow. At this time, the IVUS sensor 12a transmits and receives an ultrasonic wave at each rotation angle. Lines 1, 2, . . . 512 indicate transmission/reception directions of ultrasonic waves at each rotation angle. In the present embodiment, the IVUS sensor 12a intermittently transmits and receives ultrasonic waves 512 times while rotating 360 degrees corresponding to 1 rotation in the blood vessel. Since the IVUS sensor 12a acquires data of one line in the transmission/reception direction by transmitting and receiving an ultrasonic wave once, it is possible to obtain 512 pieces of ultrasonic line data radially extending from the rotation center during one rotation. The 512 pieces of ultrasonic line data are dense in the vicinity of the rotation center, but become sparse with distance from the rotation center. Therefore, the image processing apparatus 3 can generate a two-dimensional ultrasonic tomographic image (i.e., an IVUS image) as illustrated in FIG. 4A by generating pixels in an empty space of each line by known interpolation processing.

Similarly, the OCT sensor 12b also transmits and receives the measurement light at each rotation angle. Since the OCT sensor 12b also transmits and receives the measurement light 512 times while rotating 360 degrees in the blood vessel, it is possible to obtain 512 pieces of optical line data radially extending from the rotation center during one rotation. Moreover, for the optical line data, the image processing apparatus 3 can generate a two-dimensional optical coherence tomographic image (i.e., an OCT image) similar to the IVUS image illustrated in FIG. 4A by generating pixels in an empty space of each line by known interpolation processing. That is, the image processing apparatus 3 generates optical line data based on interference light generated by causing reflected light and, for example, reference light obtained by separating light from a light source in the image processing apparatus 3 to interfere with each other, and generates an optical coherence tomographic image (i.e., an OCT image) obtained by imaging the transverse section of the blood vessel based on the generated optical line data.

The two-dimensional tomographic image generated from the 512 pieces of line data in this manner is referred to as an IVUS image or an OCT image of one frame. Note that, since the sensor unit 12 scans while moving in the blood vessel, an IVUS image or an OCT image of one frame is acquired at each position rotated once within a movement range. That is, since the IVUS image or the OCT image of one frame is acquired at each position from the distal end side to the proximal end side of the probe 11 in the movement range, as illustrated in FIG. 4B, the IVUS image or the OCT image of a plurality of frames is acquired within the movement range.

The image diagnosis catheter 1 has a marker that does not transmit X-rays in order to confirm a positional relationship between the IVUS image obtained by the IVUS sensor 12a or the OCT image obtained by the OCT sensor 12b and the angiographic image obtained by the angiography apparatus 102. In the example illustrated in FIG. 2, a marker 14a is provided at the distal portion of the catheter sheath 11a, for example, the guide wire insertion portion 14, and a marker 12c is provided on the shaft 13 side of the sensor unit 12. When the image diagnosis catheter 1 described above is imaged with X-rays, an angiographic image in which the markers 14a and 12c are visualized is obtained. The positions at which the markers 14a and 12c are provided are an example, the marker 12c may be provided on the shaft 13 instead of the sensor unit 12, and the marker 14a may be provided at a portion other than the distal portion of the catheter sheath 11a.

FIG. 5 is a block diagram illustrating the image processing apparatus 3. The image processing apparatus 3 is an information processing device such as a computer, and includes a control unit 31, a main storage unit 32 (or a main memory), an input/output unit 33, a communication unit 34, an auxiliary storage unit 35, and a reading unit 36. The image processing apparatus 3 is not limited to a single computer, and may be formed by a plurality of computers. In addition, the image processing apparatus 3 may be a server client system, a cloud server, or a virtual machine operating as software. In the following description, it is assumed that the image processing apparatus 3 is a single computer.

The control unit 31 includes one or a plurality of arithmetic processing apparatuses such as a central processing unit (CPU), a micro processing unit (MPU), a graphics processing unit (GPU), a general purpose computing on graphics processing unit (GPGPU), and/or a tensor processing unit (TPU). The control unit 31 is connected to each hardware unit of the image processing apparatus 3 via a bus.

The main storage unit 32, which is a temporary memory area such as a static random access memory (SRAM), a dynamic random access memory (DRAM), or a flash memory, temporarily stores data necessary for the control unit 31 to execute arithmetic processing.

The input/output unit 33 includes an interface circuit that connects external apparatuses such as the intravascular inspection apparatus 101, the angiography apparatus 102, the display apparatus 4, and the input apparatus 5. The control unit 31 acquires an IVUS image and an OCT image from the intravascular inspection apparatus 101 via the input/output unit 33, and acquires an angiographic image from the angiography apparatus 102. In addition, the control unit 31 outputs a medical image signal of an IVUS image, an OCT image, or an angiographic image to the display apparatus 4 via the input/output unit 33, thereby displaying the medical image on the display apparatus 4. Furthermore, the control unit 31 receives information input to the input apparatus 5 via the input/output unit 33.

The communication unit 34 includes, for example, a communication interface circuit conforming to a communication standard such as 4G, 5G, or WiFi. The image processing apparatus 3 communicates with an external server such as a cloud server connected to an external network such as the Internet via the communication unit 34. The control unit 31 may access an external server via the communication unit 34 and refer to various data stored in a storage of the external server. Furthermore, the control unit 31 may cooperatively perform the process in the present embodiment by performing, for example, inter-process communication with the external server.

The auxiliary storage unit 35 is a storage device such as a hard disk or a solid state drive (SSD). The auxiliary storage unit 35 stores a computer program executed by the control unit 31 and various data necessary for processing of the control unit 31. Note that the auxiliary storage unit 35 may be an external storage apparatus connected to the image processing apparatus 3. The computer program executed by the control unit 31 may be written in the auxiliary storage unit 35 at the manufacturing stage of the image processing apparatus 3, or the computer program distributed by a remote server apparatus may be acquired by the image processing apparatus 3 through communication and stored in the auxiliary storage unit 35. The computer program may be readably recorded in a recording medium RM such as a magnetic disk, an optical disk, or a semiconductor memory, or may be read from the recording medium RM by the reading unit 36 and stored in the auxiliary storage unit 35. An example of the computer program stored in the auxiliary storage unit 35 is an onset risk prediction program PG for causing a computer to execute processing of predicting the onset risk of ischemic heart disease for a vascular lesion candidate.

Furthermore, the auxiliary storage unit 35 may store various computer learning models. The learning model is described by definition information. The definition information of the learning model includes information of layers constituting the learning model, information of nodes constituting each layer, and internal parameters such as a weight coefficient and a bias between nodes. The internal parameters are learned by a predetermined learning algorithm. The auxiliary storage unit 35 stores definition information of a learning model including trained internal parameters. An example of the learning model stored in the auxiliary storage unit 35 is the learning model MD1 learned to output information regarding the onset risk of ischemic heart disease when morphological information of a lesion candidate is input. The configuration of the learning model MD1 will be described in detail later.

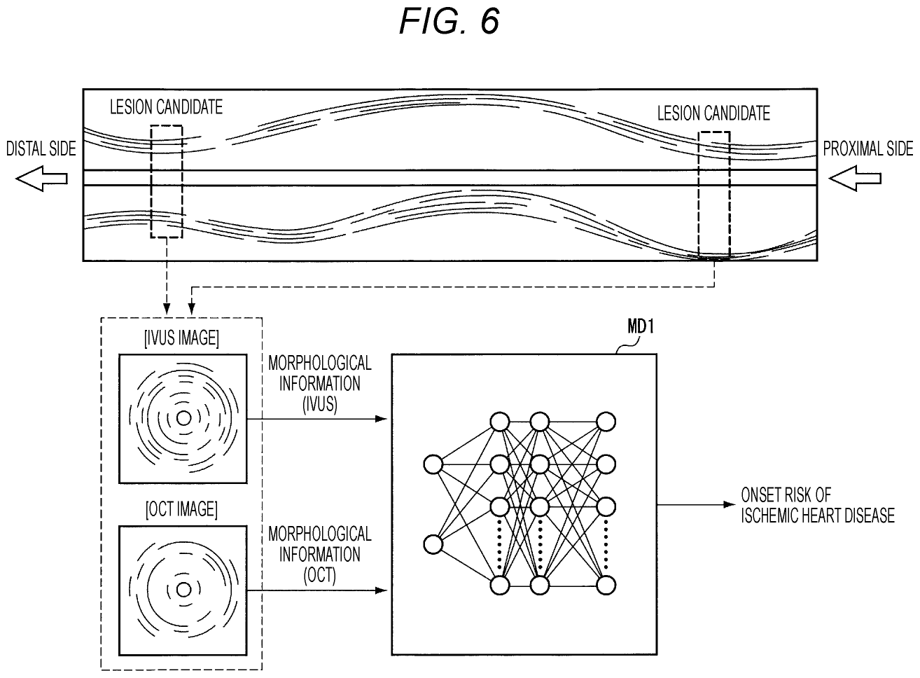

FIG. 6 is a diagram for explaining a process executed by the image processing apparatus 3. The control unit 31 of the image processing apparatus 3 specifies a lesion candidate in a blood vessel. If lipid rich structures called plaques are deposited in the walls of blood vessels (e.g., coronary arteries), ischemic heart disease such as angina pectoris and myocardial infarction may occur. The ratio of the plaque area to the cross-sectional area of the blood vessel (hereinafter referred to as plaque burden) is one of indices for specifying a lesion candidate in the blood vessel. When acquiring the IVUS image from the intravascular inspection apparatus 101, the control unit 31 can specify a lesion candidate by calculating plaque burden. Specifically, the control unit 31 calculates plaque burden from the IVUS image, and when the calculated plaque burden exceeds a preset threshold value (for example, 50%), the plaque may be specified as a lesion candidate. The example of FIG. 6 illustrates a state in which, as a result of acquiring an IVUS image while moving the sensor unit 12 of the image diagnosis catheter 1 from the distal end side (or the proximal side) to the proximal end side (or the distal side) by a pull-back operation, lesion candidates are specified at a total of two positions on the proximal side and the distal side.

The method for specifying a lesion candidate is not limited to the method for calculating plaque burden. For example, the control unit 31 may specify a lesion candidate using a computer learning model learned to identify a region such as a plaque region, a calcified region, or a thrombus region from the IVUS image. As the learning model, a learning model for object detection and a learning model for segmentation including a convolutional neural network (CNN), a U-net, a SegNet, a vision transformer (ViT), a single shot detector (SSD), a support vector machine (SVM), a Bayesian network, a regression tree, and the like can be used. Furthermore, the control unit 31 may specify a lesion candidate from an OCT image or an angiographic image instead of the IVUS image.

The control unit 31 extracts morphological information on the specified lesion candidate. The morphological information represents morphological information such as a volume, an area, a length, and a thickness that can change according to the degree of progression of the lesion. Although the IVUS image is lower than the OCT image in terms of the resolution of the obtained image, an image of a vascular tissue deeper than the OCT image is obtained. The control unit 31 extracts a feature amount (hereinafter also referred to as the first feature amount) related to a form such as a volume or an area of a plaque (e.g., a lipid core) or a length or a thickness of a neovessel from the IVUS image as morphological information. On the other hand, in the OCT image, only an image from the vascular lumen surface to a relatively shallow tissue can be obtained, but an image with high resolution can be obtained with respect to the lumen surface of the blood vessel. The control unit 31 can extract, from the OCT image, a feature amount (hereinafter also referred to as the second feature amount) related to a form such as a thickness of a fibrous cap and an area infiltrated by macrophages as morphological information.

The control unit 31 inputs the extracted morphological information to the learning model MD1 and executes computation by the learning model MD1 to estimate the onset risk of ischemic heart disease. Note that, in a case where a plurality of lesion candidates is specified in the specification of the lesion candidates, processing of extracting the morphological information and processing of estimating the onset risk of ischemic heart disease using the learning model MD1 may be performed for each of the lesion candidates.

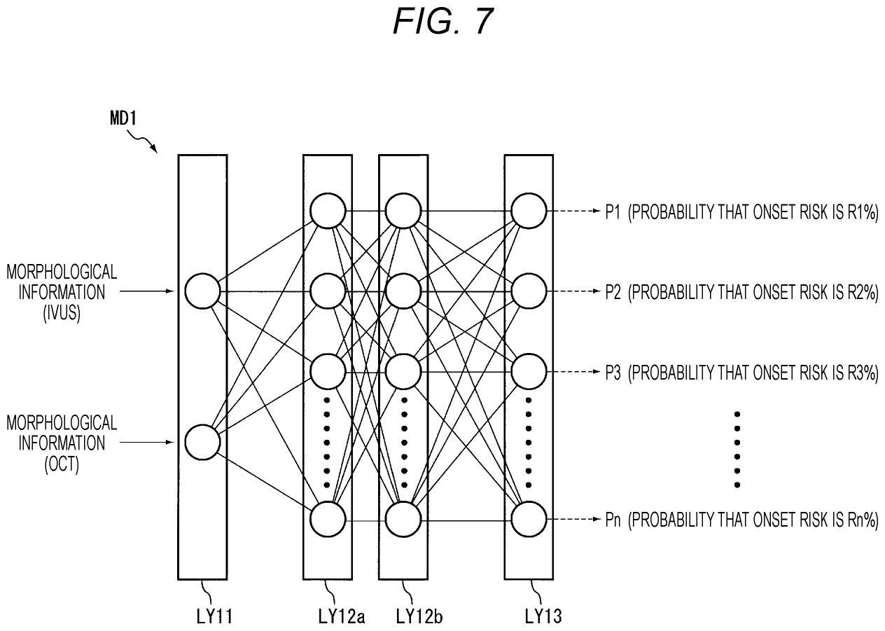

FIG. 7 is a schematic diagram illustrating a computer learning model MD1 according to the first embodiment. The learning model MD1 includes, for example, an input layer LY11, intermediate layers LY12a and 12b, and an output layer LY13. In the example of FIG. 7, one input layer LY11 is provided, but two or more input layers may be provided. In addition, in the example of FIG. 7, two intermediate layers LY12a and 12b are described, but the number of intermediate layers is not limited to two, and may be three or more. An example of the learning model MD1 is a deep neural network (DNN). Alternatively, ViT, SVM, eXtreme Gradient Boosting (XGBoost), Light Gradient Boosting Machine (LightGBM), or the like may be used.

Each layer constituting the learning model MD1 includes one or a plurality of nodes. The nodes of each layer are coupled to the nodes provided in the preceding and subsequent layers in one direction with a desired weight and bias. Vector data having the same number of components as the number of nodes of the input layer LY11 is provided as input data of the learning model MD1. The input data in the first embodiment is morphological information extracted from the IVUS image and the OCT image.

The data provided to each node of the input layer LY11 is provided to the first intermediate layer LY12a. The output is calculated in the intermediate layer LY12a using the activation function including the weight coefficient and the bias, the calculated value is given to the next intermediate layer LY12b, and the output of the output layer LY13 is successively transmitted to the subsequent layers until the output is obtained in the same manner.

The output layer LY13 outputs information related to the onset risk of ischemic heart disease. The output form of the output layer LY13 is any form. For example, n pieces (n is an integer of 1 or more) may be provided in the output layer LY13, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY13 of the learning model MD1 and estimate the highest probability as the onset risk of ischemic heart disease.

Furthermore, the learning model MD1 may be designed so as to predict the presence or absence of onset within a predetermined number of years (for example, within three years), and information of 0 (=no onset) or 1 (=onset) may be output from the output layer LY13. Furthermore, the learning model MD1 may be designed so as to calculate the probability of onset within a predetermined number of years (for example, within three years), and the probability (a real value of 0 to 1) may be output from the output layer LY13. In these cases, the number of nodes provided in the output layer LY13 may be one.

The learning model MD1 is trained according to a predetermined learning algorithm, and internal parameters (e.g., weight coefficient, bias, or the like) are determined. Specifically, it is possible to determine the internal parameters of the learning model MD1 including the weight coefficient and the bias between the nodes by using a large number of data sets including the morphological information extracted from the lesion candidate and the correct answer information indicating whether the ischemic heart disease has developed later with the lesion candidate as the culprit lesion for the training data and performing learning using an algorithm such as a backpropagation method. In the present embodiment, the trained learning model MD1 is stored in the auxiliary storage unit 35.

Note that, in the present embodiment, the information on the onset risk of ischemic heart disease (ID) is output from the learning model MD1. Alternatively, the information on the onset risk may be output only for acute coronary syndrome (ACS), or the information on the onset risk may be output only for acute myocardial infarction (AMI). In one embodiment, the information is displayed on the display apparatus 4.

Furthermore, in the present embodiment, the learning model MD1 is stored in the auxiliary storage unit 35, and the computation by the learning model MD1 is executed by the control unit 31 of the image processing apparatus 3. However, the learning model MD1 may be installed in an external server, and the external server may be accessed via the communication unit 34 to cause the external server to execute the computation by the learning model MD1. In this case, the control unit 31 of the image processing apparatus 3 may transmit the morphological information extracted from the IVUS image and the OCT image from the communication unit 34 to the external server, acquire the computation result by the learning model MID by communication, and estimate the onset risk of ischemic heart disease.

Furthermore, in the present embodiment, the onset risk of a disease at a certain timing is estimated on the basis of the morphological information extracted from the IVUS image and the OCT image captured at the certain timing. However, the time series transition of the onset risk of a disease may be derived by extracting morphological information at each timing from the IVUS image and the OCT image captured at a plurality of timings and inputting the morphological information to the learning model MD1. As a learning model for deriving the time series transition, a recurrent neural network such as seq2seq (sequence to sequence), XGBoost, LightGBM, or the like can be used. The learning model for deriving the time series transition is generated by learning using a data set including IVUS images and OCT images captured at a plurality of timings and correct answer information indicating whether an ischemic heart disease is developed in the IVUS images and the OCT images as training data.

Hereinafter, the operation of the image processing apparatus 3 will be described.

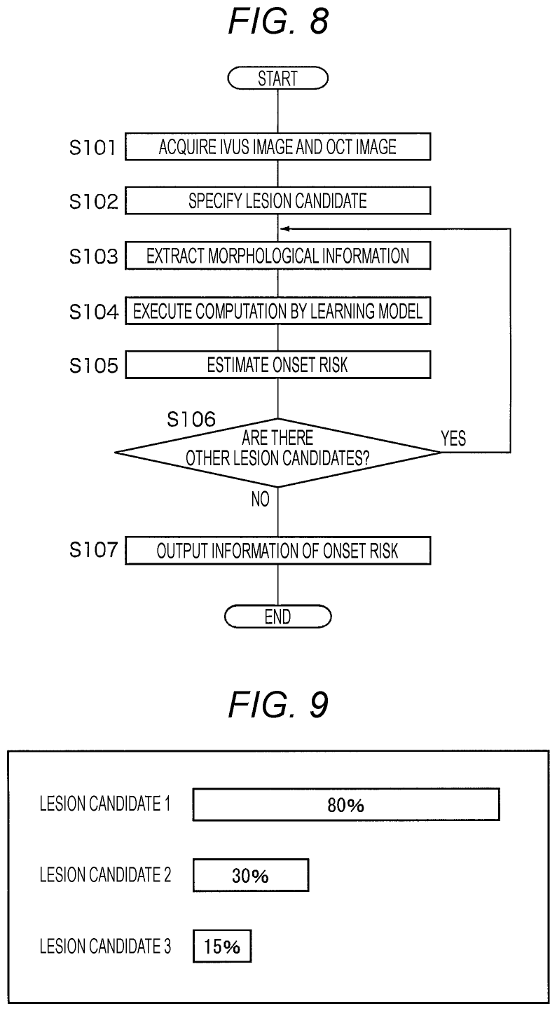

FIG. 8 is a flowchart for explaining a process executed by the image processing apparatus 3 in the first embodiment. The control unit 31 of the image processing apparatus 3 performs the following process by executing the onset risk prediction program PG stored in the auxiliary storage unit 35 in the operation phase after completing the learning of the learning model MD1. The control unit 31 acquires the IVUS image and the OCT image captured by the intravascular inspection apparatus 101 through the input/output unit 33 (S101). In the present embodiment, while the probe 11 (i.e., the image diagnosis catheter 1) is moved from the distal end side (or the proximal side) to the proximal end side (or the distal side) by a pull-back operation, the inside of the blood vessel is continuously imaged at predetermined time intervals to generate an IVUS image and an OCT image. The control unit 31 may acquire the IVUS image and the OCT image sequentially in frames, or may acquire the generated IVUS image and OCT image after the IVUS image and OCT image including a plurality of frames are generated by the intravascular inspection apparatus 101.

In addition, the control unit 31 may acquire an IVUS image and an OCT image captured for a patient before onset in order to estimate the onset risk of ischemic heart disease, and may acquire an IVUS image and an OCT image captured for follow-up after treatment such as percutaneous coronary intervention (PCI) in order to estimate the risk of re-onset of ischemic heart disease. Furthermore, in order to derive the time series transition of the onset risk, IVUS images and OCT images captured at a plurality of timings may be acquired. Furthermore, the control unit 31 may acquire an angiographic image from the angiography apparatus 102 in addition to the IVUS image and the OCT image.

The control unit 31 specifies a lesion candidate for the blood vessel of the patient (S102). For example, the control unit 31 calculates plaque burden from the IVUS image, and determines whether the calculated plaque burden exceeds a preset threshold value (for example, 50%), thereby specifying a lesion candidate. Alternatively, the control unit 31 may specify a lesion candidate using a learning model learned to identify a region such as a calcified region or a thrombus region from an IVUS image, an OCT image, or an angiographic image. In S102, one or a plurality of lesion candidates may be specified.

The control unit 31 extracts morphological information on the specified lesion candidate (S103). The control unit 31 extracts a feature amount (i.e., a first feature amount) related to the form of a lesion candidate such as an attenuated plaque (e.g., a lipid core), a remodeling index, a calcified plaque, a neovessel, or a plaque volume from the IVUS image. Here, the remodeling index is an index calculated by the vessel cross-sectional area of the lesion/((vessel cross-sectional area of proximal target site+vessel cross-sectional area of distal target site)/2). This index is an index focusing on the fact that the risk of a lesion with a bulging outer diameter of the blood vessel is high as the plaque volume increases. Note that the proximal target site represents a relatively normal site on the proximal side of the lesion, and the distal target site represents a relatively normal site on the distal side of the lesion. In addition, the control unit 31 extracts, from the OCT image, a feature amount (i.e., a second feature amount) related to the form of a lesion candidate such as the thickness of a fibrous cap, a neovessel, a calcified plaque, a lipid plaque, or infiltration of macrophages.

The control unit 31 inputs the extracted morphological information to the learning model MD1 and executes computation by the learning model MID (S104). The control unit 31 gives the first feature amount and the second feature amount to the nodes provided in the input layer LY11 of the learning model MD1, and sequentially executes the computation in the intermediate layer LY12 according to the trained internal parameters (e.g., the weight coefficient and bias). The computation result by the learning model MD1 is output from each node of the output layer LY13.

The control unit 31 refers to the information output from the output layer LY13 of the learning model MD1 and estimates the onset risk of ischemic heart disease (S105). For example, since information regarding the probability of the onset risk is output from each node of the output layer LY13, the control unit 31 can estimate the onset risk by selecting a node having the highest probability. The control unit 31 may derive the time series transition of the onset risk by extracting morphological information from the IVUS image and the OCT image captured at a plurality of timings, inputting the morphological information at each timing to the learning model MD1, and performing computation.

The control unit 31 determines whether there are other specified lesion candidates (S106). When it is determined that there is another specified lesion candidate (S106: YES), the control unit 31 returns the process to S103.

When it is determined that there are no other specified lesion candidates (S106: NO), the control unit 31 outputs information on the onset risk estimated in S105 (S107).

Note that, in the flowchart of FIG. 8, the steps of S103 to S105 are executed for each lesion candidate to estimate the onset risk. However, in a case where a plurality of lesion candidates is specified in S102, the steps of S103 to S105 may be collectively executed for all lesion candidates. In this case, it is not necessary to repeat the steps for each lesion candidate, so that the process speed is expected to be improved.

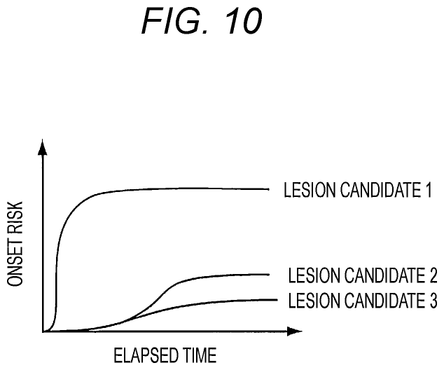

FIGS. 9 and 10 are schematic diagrams illustrating output examples of the onset risk. As illustrated in FIG. 9, the control unit 31 generates a graph indicating the level of the onset risk for each lesion candidate and causes the display apparatus 4 to display the generated graph. In addition, as illustrated in FIG. 10, the control unit 31 may generate a graph indicating a time series transition of the onset risk for each lesion candidate and display the generated graph on the display apparatus 4. Furthermore, in FIGS. 9 and 10, the level of the onset risk for each of “lesion candidate 1” to “lesion candidate 3” is indicated by a graph. However, in order to clearly indicate which part of the blood vessel corresponds to each lesion candidate, a marker may be added to a longitudinal tomographic image or an angiographic image of the blood vessel and displayed together with the graph. Instead of displaying the graph on the display apparatus 4, the control unit 31 may notify an external terminal or an external server of information on the onset risk (e.g., numerical information or graph) through the communication unit 34.

As described above, in the first embodiment, the morphological information is extracted from both the IVUS image and the OCT image, and the onset risk of ischemic heart disease is estimated on the basis of the extracted morphological information. Therefore, it is possible to accurately estimate the onset risk of ischemic heart disease, which is conventionally considered difficult.

In particular, it is known that myocardial infarction is more likely to re-develop due to a non-culprit lesion than due to a culprit lesion. The culprit lesion is a lesion caused by the onset of ischemic heart disease, and treatment such as PCI is performed as necessary. On the other hand, the non-culprit lesion is a lesion not caused by the onset of ischemic heart disease, and treatment such as PCI is rarely performed. According to the above procedure, when it is estimated that the onset risk of ischemic heart disease is high from the IVUS image and the OCT image acquired after treatment such as PCI (that is, when it is estimated that the risk of re-onset of the disease is high), the risk of re-onset can be reduced by performing treatment such as PCI on the corresponding lesion candidate.

### Second Embodiment

In a second embodiment, a configuration for directly estimating the onset risk of ischemic heart disease from an IVUS image and an OCT image will be described.

Since the overall configuration of the image diagnosis system 100, the internal configuration of the image processing apparatus 3, and the like are similar to those of the first embodiment, the description thereof will be omitted.

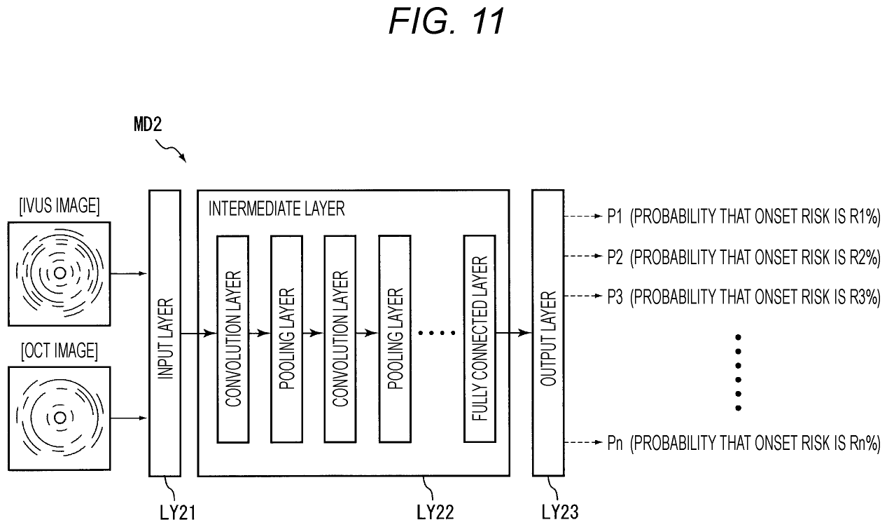

FIG. 11 is a schematic diagram illustrating a computer learning model MD2 in the second embodiment. The learning model MD2 includes, for example, an input layer LY21, an intermediate layer LY22, and an output layer LY23. An example of the learning model MD2 is a learning model based on CNN. Alternatively, the learning model MD2 may be a learning model based on a region-based CNN (R-CNN), a You Only Look Once (YOLO), an SSD, an SVM, a decision tree, or the like.

An IVUS image and an OCT image are input to the input layer LY21. Data of the IVUS image and the OCT image input to the input layer LY21 is provided to the intermediate layer LY22.

The intermediate layer LY22 includes a convolution layer, a pooling layer, a fully connected layer, and the like. A plurality of convolution layers and a plurality of pooling layers may be alternately provided. The convolution layer and the pooling layer extract features of the IVUS image and the OCT image input from the input layer LY21 by computation using nodes of the respective layers. The fully connected layer connects the data in which the feature portion is extracted by the convolution layer and the pooling layer to one node, and outputs the feature variable converted by the activation function. The feature variable is output to the output layer through the fully connected layer.

The output layer LY23 includes one or more nodes. The output form of the output layer LY23 is any form. For example, the output layer LY23 calculates a probability for each onset risk of ischemic heart disease based on the feature variable input from the fully connected layer of the intermediate layer LY22, and outputs the probability from each node. In this case, n pieces (n is an integer of 1 or more) may be provided in the output layer LY23, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY23 of the learning model MD2 and estimate the highest probability as the onset risk of ischemic heart disease.

Furthermore, the learning model MD2 may be designed so as to predict the presence or absence of onset within a predetermined number of years (for example, within three years), and information of 0 (=no onset) or 1 (=onset) may be output from the output layer LY23. Furthermore, the learning model MD2 may be designed so as to calculate the probability of onset within a predetermined number of years (for example, within three years), and the probability (a real value of 0 to 1) may be output from the output layer LY23. In these cases, the number of nodes provided in the output layer LY23 may be one.

In the second embodiment, when acquiring the IVUS image and the OCT image captured by the intravascular inspection apparatus 101, the control unit 31 of the image processing apparatus 3 inputs the acquired IVUS image and OCT image to the learning model MD2 and executes computation using the learning model MD2. The control unit 31 estimates the onset risk of ischemic heart disease with reference to the information output from the output layer LY23 of the learning model MD2.

As described above, in the second embodiment, since both the IVUS image and the OCT image are input to the learning model MD2 to estimate the onset risk of ischemic heart disease, it is possible to accurately estimate the onset risk of ischemic heart disease, which is conventionally considered difficult.

In the example of FIG. 11, the IVUS image and the OCT image are input to the input layer LY21, and the feature variable is derived in the intermediate layer LY22. However, the learning model MD2 may include a first input layer to which the IVUS image is input, a first intermediate layer that derives the feature variable from the IVUS image input to the first input layer, a second input layer to which the OCT image is input, and a second intermediate layer that derives the feature variable from the OCT image input to the second input layer. In this case, in the output layer, the final probability may be calculated based on the feature variable output from the first intermediate layer and the feature variable output from the second intermediate layer.

### Third Embodiment

In a third embodiment, a configuration will be described in which a value of stress applied to a lesion candidate is calculated, and the onset risk of ischemic heart disease is estimated based on the calculated value of stress.

Since the overall configuration of the image diagnosis system 100, the internal configuration of the image processing apparatus 3, and the like are similar to those of the first embodiment, the description thereof will be omitted.

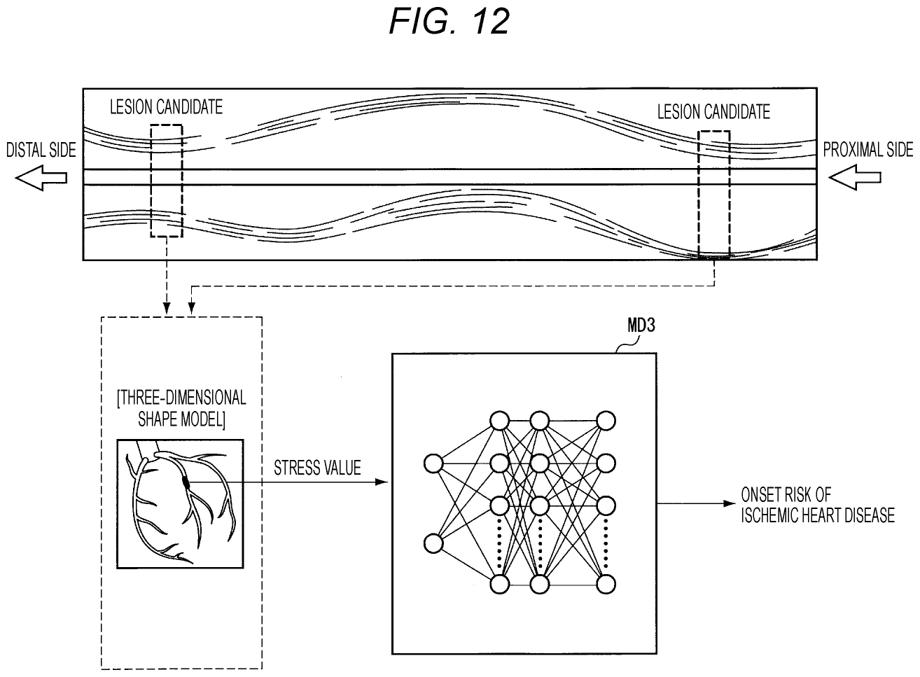

FIG. 12 is a diagram for explaining a process executed in the third embodiment. The control unit 31 of the image processing apparatus 3 specifies a lesion candidate in a blood vessel. The method of specifying a lesion candidate is similar to that in the first embodiment. For example, the control unit 31 calculates plaque burden from an IVUS image, and if the calculated plaque burden exceeds a preset threshold value (for example, 50%), the plaque may be specified as a lesion candidate. Furthermore, the control unit 31 may specify a lesion candidate using a learning model for object detection or a learning model for segmentation, or may specify a lesion candidate from an OCT image or an angiographic image.

The control unit 31 calculates a value of stress applied to the specified lesion candidate. For example, the shear stress and the normal stress applied to the lesion candidate can be calculated by simulation using a three-dimensional shape model of a blood vessel. The three-dimensional shape model can be generated based on voxel data obtained by regenerating a tomographic CT image or an MRI image. The shear stress applied to the wall surface of the blood vessel is calculated using, for example, Formula 1.

$${τ}_{w} = - \frac{r}{2} \frac{dp}{dx} \qquad [ Math . 1 ]$$

Here, rw represents the shear stress applied to the lesion candidate (i.e., the wall surface of the blood vessel), r represents the radius of the blood vessel, and dp/dx represents the pressure gradient in the length direction of the blood vessel. Formula 1 is derived based on the balance between the action force of the pressure loss caused by the friction loss of the blood vessel and the frictional force caused by the shear stress. The control unit 31 may calculate the maximum value of the shear stress applied to the lesion candidate using, for example, Formula 1, or may calculate the average value.

The shear stress may vary depending on the structure or shape of the blood vessel and the state of blood flow. Therefore, the control unit 31 simulates the blood flow using the three-dimensional shape model of the blood vessel and derives the loss coefficient of the blood vessel, thereby calculating the shear stress applied to the lesion candidate. Similarly, the control unit 31 can calculate the normal stress applied to the lesion candidate by simulating the blood flow using the three-dimensional shape model of the blood vessel. The normal stress applied to the wall surface of the blood vessel is calculated using, for example, Formula 2.

$$σ = - p - \frac{2}{3} μ div \vec{v} + 2 μ \frac{\partial u}{\partial x} \qquad [ Math . 2 ]$$

Here, a represents a normal stress applied to a lesion candidate (i.e., a wall surface of a blood vessel), p represents a pressure, represents a viscosity coefficient, v represents a velocity of blood flow, and x represents a displacement of a fluid element. The control unit 31 may calculate the maximum value of the normal stress applied to the lesion candidate using, for example, Formula 2, or may calculate the average value.

Note that the method of calculating the shear stress and the normal stress applied to the lesion candidate is not limited to those described above. For example, a method disclosed in a paper such as “Intravascular Ultrasound-Derived Virtual Fractional Flow Reserve for the Assessment of Myocardial Ischemia, Fumiyasu Seike et. al, Circ J 2018; 82: 815-823” or “Intracoronary Optical Coherence Tomography-Derived Virtual Fractional Flow Reserve for the Assessment of Coronary Artery Disease, Fumiyasu Seike el. al, Am J Cardiol. 2017 Nov. 15; 120(10): 1772-1779” may be used. In addition, without using the three-dimensional shape model of the blood vessel, the shape and blood flow of the blood vessel may be calculated from the IVUS image, the OCT image, and the angiographic image, and the value of stress (e.g., a pseudo value) may be calculated using the calculated shape and blood flow.

The control unit 31 inputs the calculated stress value to the learning model MD3 and executes computation by the learning model MD3 to estimate the onset risk of ischemic heart disease. Note that, in a case where a plurality of lesion candidates is specified in the specification of the lesion candidates, processing of calculating a stress value and processing of estimating the onset risk of ischemic heart disease using the learning model MD3 may be performed for each of the lesion candidates.

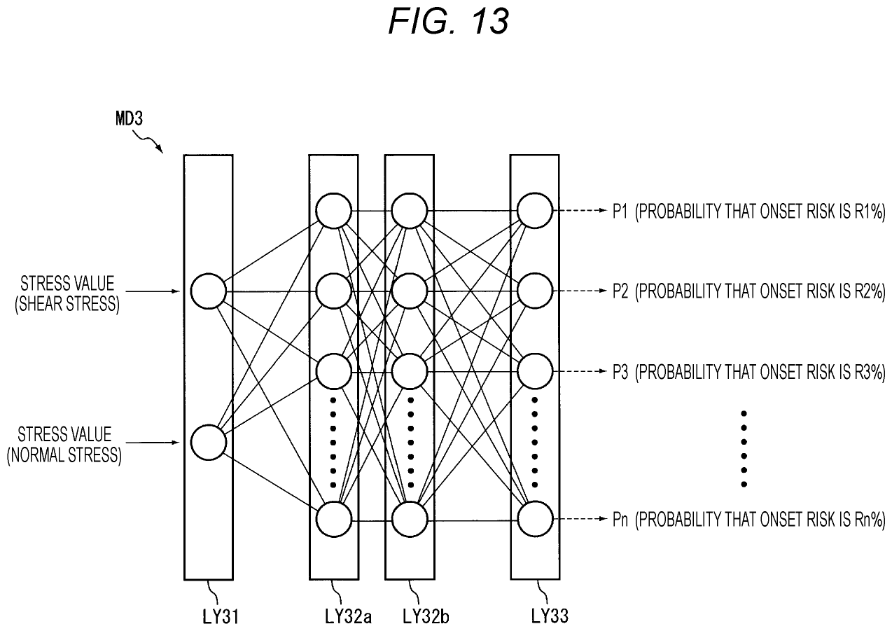

FIG. 13 is a schematic diagram illustrating a computer learning model MD3 in the third embodiment. The configuration of the learning model MD3 is similar to that of the first embodiment, and includes an input layer LY31, intermediate layers LY32a and 32b, and an output layer LY33. An example of the learning model MD3 is a DNN. Alternatively, SVM, XGBoost, LightGBM, or the like is used.

The input data in the third embodiment is a value of stress applied to a lesion candidate. Both the shear stress and the normal stress may be input to the input layer LY31, or only one of the values may be input to the input layer LY31.

The data provided to each node of the input layer LY31 is provided to the first intermediate layer LY32a. The output is calculated in the intermediate layer LY32a using the activation function including the weight coefficient and the bias, the calculated value is given to the next intermediate layer LY32b, and the output of the output layer LY33 is successively transmitted to the subsequent layers until the output is obtained in the same manner.

The output layer LY33 outputs information related to the onset risk of ischemic heart disease. The output form of the output layer LY33 is any form. For example, n pieces (n is an integer of 1 or more) may be provided in the output layer LY33, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY33 of the learning model MD3 and estimate the highest probability as the onset risk of ischemic heart disease.

Furthermore, the learning model MD3 may be designed so as to predict the presence or absence of onset within a predetermined number of years (for example, within three years), and information of 0 (=no onset) or 1 (=onset) may be output from the output layer LY33. Furthermore, the learning model MD3 may be designed so as to calculate the probability of onset within a predetermined number of years (for example, within three years), and the probability (a real value of 0 to 1) may be output from the output layer LY33. In these cases, the number of nodes provided in the output layer LY33 may be one.

The learning model MD3 is trained according to a predetermined learning algorithm, and internal parameters (e.g., weight coefficient, bias, or the like) are determined. Specifically, it is possible to determine the internal parameters of the learning model MD3 including the weight coefficient and the bias between the nodes by using a large number of data sets including the value of the stress calculated for the lesion candidate and the correct answer information indicating whether the ischemic heart disease has developed later with the lesion candidate as the culprit lesion for the training data and performing learning using an algorithm such as a backpropagation method. In the present embodiment, the trained learning model MD3 is stored in the auxiliary storage unit 35.

Note that, in the present embodiment, the information on the onset risk of ischemic heart disease (ID) is output from the learning model MD3. Alternatively, the information on the onset risk may be output only for acute coronary syndrome (ACS), or the information on the onset risk may be output only for acute myocardial infarction (AMI).

In addition, the learning model MD3 may be installed in an external server, and the external server may be accessed via the communication unit 34 to cause the external server to execute the computation by the learning model MD3.

Furthermore, the control unit 31 may derive the time series transition of the onset risk by inputting the values of stress calculated at a plurality of timings to the learning model MD3.

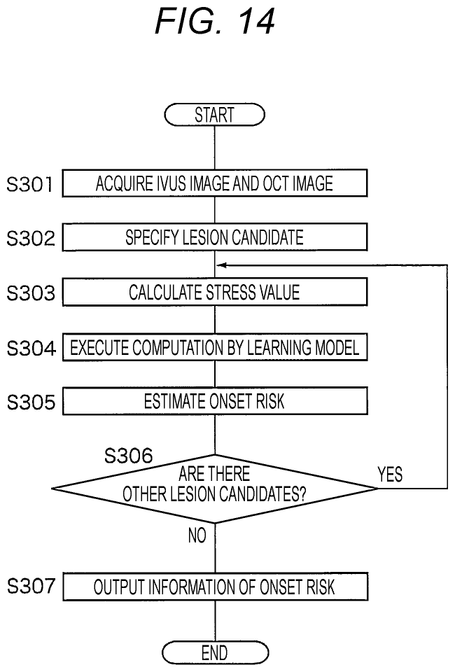

FIG. 14 is a flowchart for explaining a process executed by the image processing apparatus 3 in the third embodiment. The control unit 31 of the image processing apparatus 3 executes the onset risk prediction program PG stored in the auxiliary storage unit 35 to perform the following process. The control unit 31 acquires the IVUS image and the OCT image captured by the intravascular inspection apparatus 101 through the input/output unit 33 (S301).

The control unit 31 specifies a lesion candidate for the blood vessel of the patient (S302). For example, the control unit 31 calculates plaque burden from the IVUS image, and determines whether the calculated plaque burden exceeds a preset threshold value (for example, 50%), thereby specifying a lesion candidate. Alternatively, the control unit 31 may specify a lesion candidate using a learning model learned to identify a region such as a calcified region or a thrombus region from an IVUS image, an OCT image, or an angiographic image. In S302, one or a plurality of lesion candidates may be specified.

The control unit 31 calculates a value of stress applied to the specified lesion candidate (S303). The control unit 31 can calculate the value of the stress applied to the lesion candidate by performing simulation using the three-dimensional shape model of the blood vessel. Specifically, the control unit 31 may calculate the shear stress by Formula 1 and the normal stress by Formula 2.

The control unit 31 inputs the calculated value of the stress to the learning model MD3 and executes computation by the learning model MD3 (S304). The control unit 31 gives values of the shear stress and the normal stress to the nodes provided in the input layer LY31 of the learning model MD3, and sequentially executes the computation in the intermediate layer LY32 according to the trained internal parameters (e.g., weight coefficient and bias). The computation result by the learning model MD3 is output from each node of the output layer LY33.

The control unit 31 refers to the information output from the output layer LY33 of the learning model MD3 and estimates the onset risk of ischemic heart disease (S305). For example, since information regarding the probability of the onset risk is output from each node of the output layer LY33, the control unit 31 can estimate the onset risk by selecting a node having the highest probability. The control unit 31 may derive the time series transition of the onset risk by inputting the values of stress calculated at a plurality of timings to the learning model MD3 and performing computation.

The control unit 31 determines whether there are other specified lesion candidates (S306). When it is determined that there is another specified lesion candidate (S306: YES), the control unit 31 returns the process to S303.

When it is determined that there are no other specified lesion candidates (S306: NO), the control unit 31 outputs information on the onset risk estimated in S305 (S307). The output method is similar to that of the first embodiment. For example, as illustrated in FIG. 9, a graph indicating the level of the onset risk for each lesion candidate may be generated and displayed on the display apparatus 4, or a graph indicating the time series transition of the onset risk for each lesion candidate as illustrated in FIG. 10 may be generated and displayed on the display apparatus 4. Alternatively, the control unit 31 may notify the external terminal or the external server of the information on the onset risk through the communication unit 34.

As described above, in the third embodiment, the value of stress applied to the lesion candidate is calculated, and the onset risk of ischemic heart disease is estimated on the basis of the calculated value of stress. Therefore, it is possible to accurately estimate the onset risk of ischemic heart disease, which is conventionally considered difficult.

### Fourth Embodiment

In a fourth embodiment, a configuration for estimating the onset risk of ischemic heart disease based on morphological information extracted from a lesion candidate and a value of stress calculated for the lesion candidate will be described.

Since the overall configuration of the image diagnosis system 100, the internal configuration of the image processing apparatus 3, and the like are similar to those of the first embodiment, the description thereof will be omitted.

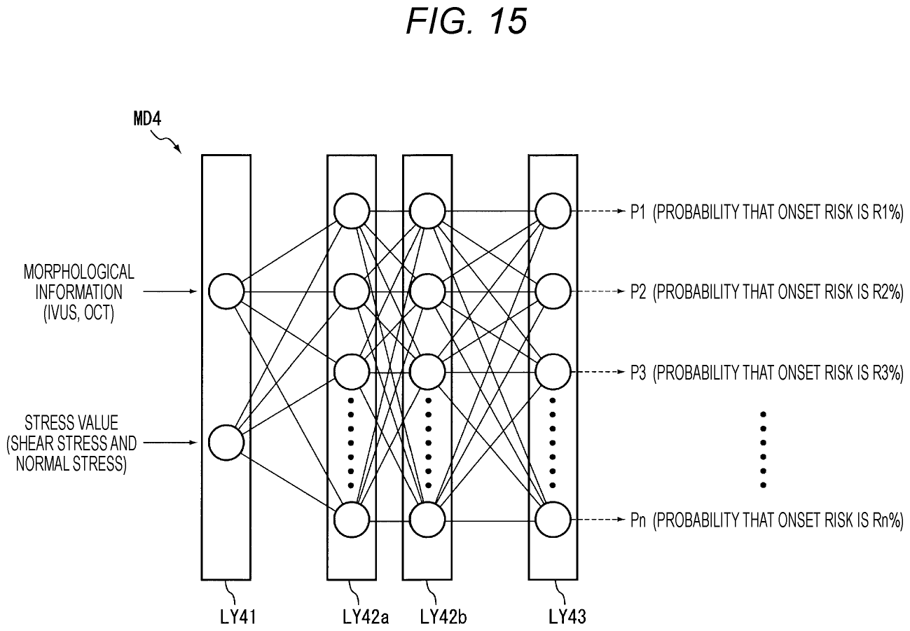

FIG. 15 is a schematic diagram illustrating a computer learning model MD4 in the fourth embodiment. The configuration of the learning model MD4 is similar to that of the first embodiment, and includes an input layer LY41, intermediate layers LY42a and 42b, and an output layer LY43. An example of the learning model MD4 is a DNN. Alternatively, SVM, XGBoost, LightGBM, or the like is used.

The input data in the fourth embodiment is morphological information extracted from a lesion candidate and a value of stress applied to the lesion candidate. The method of extracting morphological information is similar to that of the first embodiment, and the control unit 31 can extract feature amounts (i.e., first feature amounts) related to forms such as attenuated plaque (e.g., a lipid core), remodeling index, calcified plaque, neovessels, and plaque volume from the IVUS image, and extract feature amounts (i.e., second feature amounts) related to forms such as the thickness of the fibrous cap, neovessels, calcified plaque, lipid plaque, and infiltration of macrophages from the OCT image. A stress calculation method is similar to that of the first embodiment, and for example, a value of stress in a lesion candidate can be calculated by simulation using a three-dimensional shape model. In the present embodiment, morphological information extracted from the IVUS image and the OCT image, and a value of stress (at least one of shear stress and normal stress) calculated for a lesion candidate are input to the input layer LY41 of the learning model MD4.

The data provided to each node of the input layer LY41 is provided to the first intermediate layer LY42a. The output is calculated in the intermediate layer LY42a using the activation function including the weight coefficient and the bias, the calculated value is given to the next intermediate layer LY42b, and the output of the output layer LY43 is successively transmitted to the subsequent layers until the output is obtained in the same manner.

The output layer LY43 outputs information related to the onset risk of ischemic heart disease. The output form of the output layer LY43 is any form. For example, n pieces (n is an integer of 1 or more) may be provided in the output layer LY43, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY43 of the learning model MD4 and estimate the highest probability as the onset risk of ischemic heart disease.

Furthermore, the learning model MD4 may be designed so as to predict the presence or absence of onset within a predetermined number of years (for example, within three years), and information of 0 (=no onset) or 1 (=onset) may be output from the output layer LY43. Furthermore, the learning model MD4 may be designed so as to calculate the probability of onset within a predetermined number of years (for example, within three years), and the probability (a real value of 0 to 1) may be output from the output layer LY43. In these cases, the number of nodes provided in the output layer LY43 may be one.

The learning model MD4 is trained according to a predetermined learning algorithm, and internal parameters (e.g., weight coefficient, bias, or the like) are determined. Specifically, it is possible to determine the internal parameters of the learning model MD4 including the weight coefficient and the bias between the nodes by using a large number of data sets including the morphological information extracted from the lesion candidate, the value of the stress calculated for the lesion candidate and the correct answer information indicating whether the ischemic heart disease has developed later with the lesion candidate as the culprit lesion for the training data and performing learning using an algorithm such as a backpropagation method. In the present embodiment, the trained learning model MD4 is stored in the auxiliary storage unit 35.

In a case where the IVUS image and the OCT image are acquired, the control unit 31 of the image processing apparatus 3 extracts the morphological information of the lesion candidate from these images. In addition, the control unit 31 calculates the value of stress in the lesion candidate using the three-dimensional shape model of the blood vessel. The control unit 31 inputs the morphological information and the value of stress to the learning model MD4 and executes computation by the learning model MD4 to estimate the onset risk of ischemic heart disease.

Note that, in the present embodiment, the information on the onset risk of ischemic heart disease (ID) is output from the learning model MD4. Alternatively, the information on the onset risk may be output only for acute coronary syndrome (ACS), or the information on the onset risk may be output only for acute myocardial infarction (AMI).

In addition, the learning model MD4 may be installed in an external server, and the external server may be accessed via the communication unit 34 to cause the external server to execute the computation by the learning model MD4.

Furthermore, the control unit 31 may derive the time series transition of the onset risk by inputting the morphological information and the values of stress extracted at a plurality of timings to the learning model MD4.

### Fifth Embodiment

In a fifth embodiment, a configuration for estimating the onset risk of ischemic heart disease based on the value of stress calculated for a lesion candidate and a tomographic image of a blood vessel will be described.

Since the overall configuration of the image diagnosis system 100, the internal configuration of the image processing apparatus 3, and the like are similar to those of the first embodiment, the description thereof will be omitted.

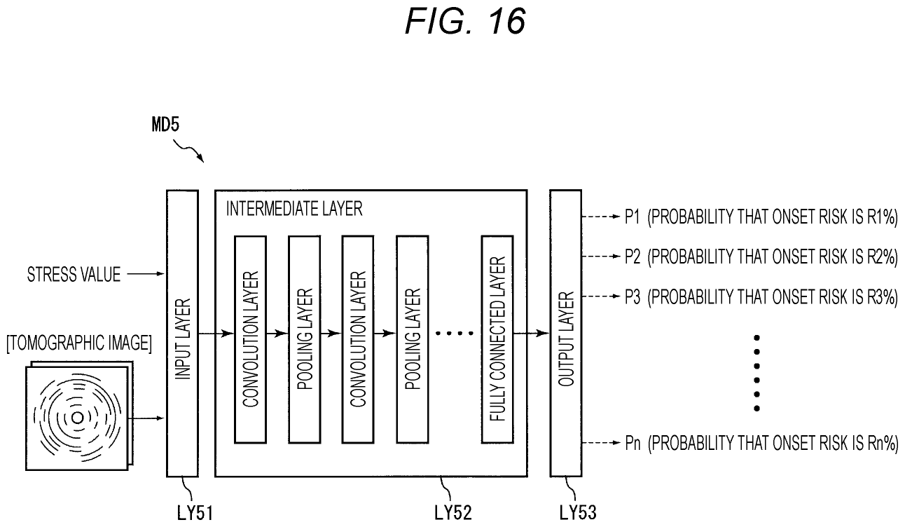

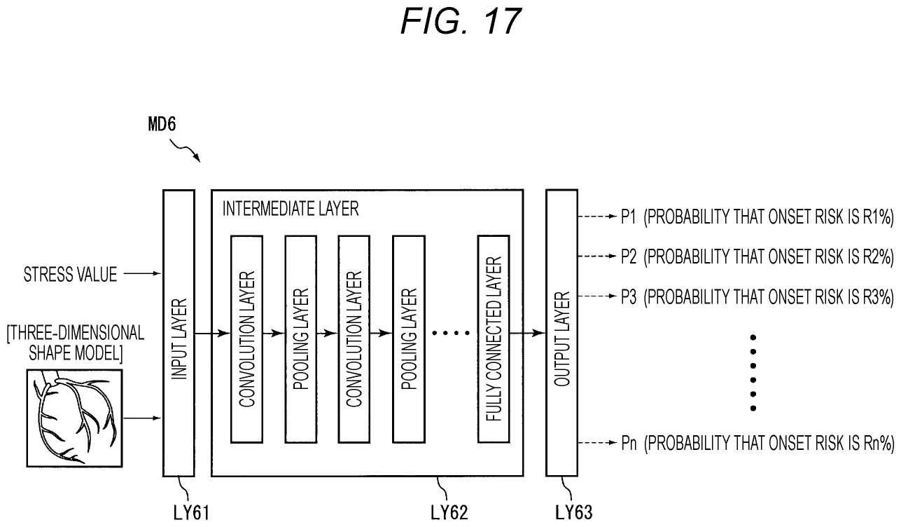

FIG. 16 is a schematic diagram illustrating a computer learning model MD5 in the fifth embodiment. The learning model MD5 includes, for example, an input layer LY51, an intermediate layer LY52, and an output layer LY53. An example of the learning model MD5 is a learning model based on CNN. Alternatively, the learning model MD5 may be a learning model based on an R-CNN, a YOLO, an SSD, an SVM, a decision tree, or the like.

The value of the stress calculated for the lesion candidate and the tomographic image of the blood vessel are input to the input layer LY51. A stress calculation method is similar to that of the first embodiment, and for example, a value of stress in a lesion candidate can be calculated by simulation using a three-dimensional shape model. The tomographic images are an IVUS image and an OCT image. The stress value and the tomographic image data input to the input layer LY51 are given to the intermediate layer LY52.

The intermediate layer LY52 includes a convolution layer, a pooling layer, a fully connected layer, and the like. A plurality of convolution layers and a plurality of pooling layers may be alternately provided. The convolution layer and the pooling layer extract the stress value input from the input layer LY51 and the feature of the tomographic image by computation using the node of each layer. The fully connected layer connects the data in which the feature portion is extracted by the convolution layer and the pooling layer to one node, and outputs the feature variable converted by the activation function. The feature variable is output to the output layer through the fully connected layer. The intermediate layer LY52 may separately include one or a plurality of hidden layers for calculating the feature variable from the stress value. In this case, the feature variable calculated from the stress value and the feature variable calculated from the tomographic image may be combined in the fully connected layer to derive the final feature variable.

The output layer LY53 includes one or more nodes. The output form of the output layer LY53 is any form. For example, the output layer LY53 calculates a probability for each onset risk of ischemic heart disease based on the feature variable input from the fully connected layer of the intermediate layer LY52, and outputs the probability from each node. For example, n pieces (n is an integer of 1 or more) may be provided in the output layer LY53, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY53 of the learning model MD5 and estimate the highest probability as the onset risk of ischemic heart disease.

Furthermore, the learning model MD5 may be designed so as to predict the presence or absence of onset within a predetermined number of years (for example, within three years), and information of 0 (=no onset) or 1 (=onset) may be output from the output layer LY53. Furthermore, the learning model MD5 may be designed so as to calculate the probability of onset within a predetermined number of years (for example, within three years), and the probability (a real value of 0 to 1) may be output from the output layer LY53. In these cases, the number of nodes provided in the output layer LY53 may be one.

In the fifth embodiment, when acquiring a tomographic image captured by the intravascular inspection apparatus 101, the control unit 31 of the image processing apparatus 3 calculates a stress value for a lesion candidate specified from the tomographic image or the like, inputs the stress value and the tomographic image to the learning model MD5, and executes computation by the learning model MD5. The control unit 31 estimates the onset risk of ischemic heart disease with reference to the information output from the output layer LY53 of the learning model MD5.

As described above, in the fifth embodiment, since the stress value and the tomographic image of the lesion candidate are input to the learning model MD5 to estimate the onset risk of ischemic heart disease, it is possible to accurately estimate the onset risk of ischemic heart disease, which is conventionally considered difficult.

### Sixth Embodiment

In a sixth embodiment, a configuration for estimating the onset risk of ischemic heart disease based on a value of stress calculated for a lesion candidate and a three-dimensional shape model of a blood vessel will be described.

Since the overall configuration of the image diagnosis system 100, the internal configuration of the image processing apparatus 3, and the like are similar to those of the first embodiment, the description thereof will be omitted.

FIG. 17 is a schematic diagram illustrating a computer learning model MD6 in the sixth embodiment. The learning model MD6 includes, for example, an input layer LY61, an intermediate layer LY62, and an output layer LY63. An example of the learning model MD5 is a learning model based on CNN. Alternatively, the learning model MD6 may be a learning model based on an R-CNN, a YOLO, an SSD, an SVM, a decision tree, or the like.

A value of stress calculated for a lesion candidate and a three-dimensional shape model of a blood vessel are input to the input layer LY61. A stress calculation method is similar to that of the first embodiment, and for example, a value of stress in a lesion candidate can be calculated by simulation using a three-dimensional shape model. The three-dimensional shape model is a model generated based on voxel data obtained by regenerating a tomographic CT image or an MRI image. The stress value input to the input layer LY61 and the data of the three-dimensional shape model are given to the intermediate layer LY62.

The intermediate layer LY62 includes a convolution layer, a pooling layer, a fully connected layer, and the like. A plurality of convolution layers and a plurality of pooling layers may be alternately provided. The convolution layer and the pooling layer extract the stress value input from the input layer LY61 and the feature of the tomographic image by computation using the node of each layer. The fully connected layer connects the data in which the feature portion is extracted by the convolution layer and the pooling layer to one node, and outputs the feature variable converted by the activation function. The feature variable is output to the output layer through the fully connected layer. The intermediate layer LY62 may separately include one or a plurality of hidden layers for calculating the feature variable from the stress value. In this case, the feature variable calculated from the stress value and the feature variable calculated from the tomographic image may be combined in the fully connected layer to derive the final feature variable.

The output layer LY63 includes one or more nodes. The output form of the output layer LY63 is any form. For example, the output layer LY63 calculates a probability for each onset risk of ischemic heart disease based on the feature variable input from the fully connected layer of the intermediate layer LY62, and outputs the probability from each node. In this case, n pieces (n is an integer of 1 or more) may be provided in the output layer LY63, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY23 of the learning model MD2 and estimate the highest probability as the onset risk of ischemic heart disease.

Furthermore, the learning model MD6 may be designed so as to predict the presence or absence of onset within a predetermined number of years (for example, within three years), and information of 0 (=no onset) or 1 (=onset) may be output from the output layer LY63. Furthermore, the learning model MD6 may be designed so as to calculate the probability of onset within a predetermined number of years (for example, within three years), and the probability (a real value of 0 to 1) may be output from the output layer LY63. In these cases, the number of nodes provided in the output layer LY63 may be one.

In the sixth embodiment, the control unit 31 of the image processing apparatus 3 calculates a stress value for a lesion candidate of a blood vessel, inputs the stress value and a three-dimensional shape model of the blood vessel to the learning model MD6, and executes computation by the learning model MD6. The control unit 31 estimates the onset risk of ischemic heart disease with reference to the information output from the output layer LY63 of the learning model MD6.

As described above, in the sixth embodiment, since the stress value and the three-dimensional shape model of the lesion candidate are input to the learning model MD6 to estimate the onset risk of ischemic heart disease, it is possible to accurately estimate the onset risk of ischemic heart disease, which is conventionally considered difficult.

### Seventh Embodiment

In a seventh embodiment, a configuration for estimating the onset risk of ischemic heart disease based on morphological information of a lesion candidate and blood inspection information will be described.

Since the overall configuration of the image diagnosis system 100, the internal configuration of the image processing apparatus 3, and the like are similar to those of the first embodiment, the description thereof will be omitted.

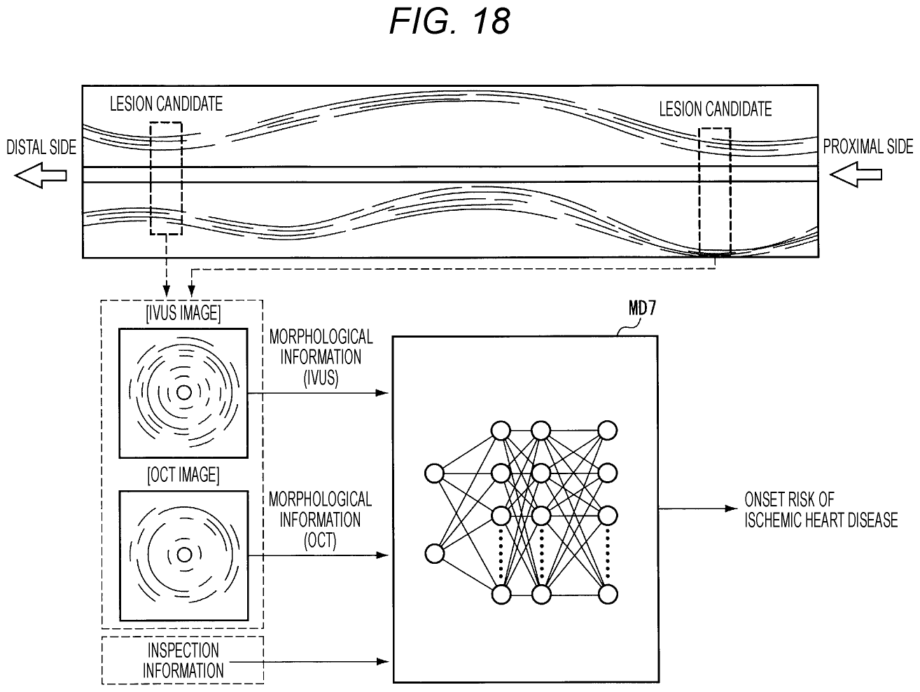

FIG. 18 is a diagram for explaining a process in the seventh embodiment. The control unit 31 of the image processing apparatus 3 specifies a lesion candidate in a blood vessel. The method of specifying a lesion candidate is similar to that in the first embodiment. For example, the control unit 31 calculates plaque burden from an IVUS image, and if the calculated plaque burden exceeds a preset threshold value (for example, 50%), the plaque may be specified as a lesion candidate. Furthermore, the control unit 31 may specify a lesion candidate using a learning model for object detection or a learning model for segmentation, or may specify a lesion candidate from an OCT image or an angiographic image.

The control unit 31 extracts morphological information on the specified lesion candidate. The method of extracting morphological information is similar to that of the first embodiment, and the control unit 31 extracts feature amounts (i.e., first feature amounts) related to forms such as attenuated plaque (e.g., a lipid core), remodeling index, calcified plaque, neovessels, and plaque volume from the IVUS image, and extracts feature amounts (i.e., second feature amounts) related to forms such as the thickness of the fibrous cap, neovessels, calcified plaque, lipid plaque, and infiltration of macrophages from the OCT image.

In the seventh embodiment, blood inspection information is further used. An example of the inspection information is a value of C-reactive protein (CRP). CRP is a protein that increases when inflammation occurs in the body or a disorder occurs in tissue cells. Alternatively, values of HDL cholesterol, LDL cholesterol, triglycerides, non-HDL cholesterol, and the like may be used. The inspection information is separately measured and input to the image processing apparatus 3 using the communication unit 34 or the input apparatus 5.

The control unit 31 inputs the extracted morphological information and the acquired inspection information to the learning model MD7 and executes computation by the learning model MD7 to estimate the onset risk of ischemic heart disease. Note that, in a case where a plurality of lesion candidates is specified in the specification of the lesion candidates, processing of extracting the morphological information and processing of estimating the onset risk of ischemic heart disease using the learning model MD7 may be performed for each of the lesion candidates.

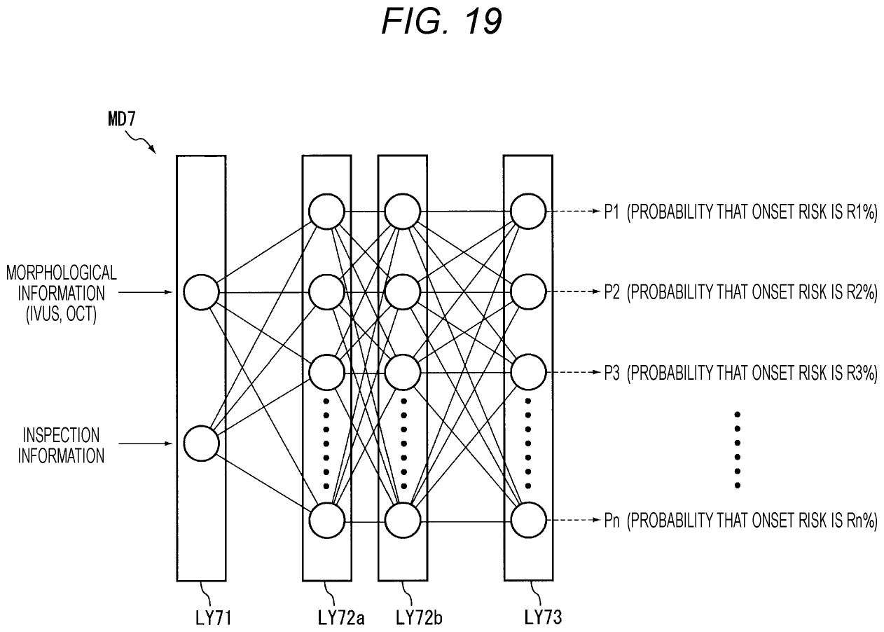

FIG. 19 is a schematic diagram illustrating a computer learning model MD7 in the seventh embodiment. The configuration of the learning model MD7 is similar to that of the first embodiment, and includes an input layer LY71, intermediate layers LY72a and 72b, and an output layer LY73. An example of the learning model MD7 is a DNN. Alternatively, SVM, XGBoost, LightGBM, or the like is used.

The input data in the seventh embodiment is morphological information of a lesion candidate and blood inspection information. The data provided to each node of the input layer LY71 is provided to the first intermediate layer LY72a. The output is calculated in the intermediate layer LY72a using the activation function including the weight coefficient and the bias, the calculated value is given to the next intermediate layer LY72b, and the output of the output layer LY73 is successively transmitted to the subsequent layers until the output is obtained in the same manner.

The output layer LY73 outputs information related to the onset risk of ischemic heart disease. The output form of the output layer LY73 is any form. For example, n pieces (n is an integer of 1 or more) may be provided in the output layer LY73, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY73 of the learning model MD7 and estimate the highest probability as the onset risk of ischemic heart disease.

Furthermore, the learning model MD7 may be designed so as to predict the presence or absence of onset within a predetermined number of years (for example, within three years), and information of 0 (=no onset) or 1 (=onset) may be output from the output layer LY73. Furthermore, the learning model MD7 may be designed so as to calculate the probability of onset within a predetermined number of years (for example, within three years), and the probability (a real value of 0 to 1) may be output from the output layer LY73. In these cases, the number of nodes provided in the output layer LY73 may be one.

The learning model MD7 is trained according to a predetermined learning algorithm, and internal parameters (e.g., weight coefficient, bias, or the like) are determined. Specifically, it is possible to determine the internal parameters of the learning model MD7 including the weight coefficient and the bias between the nodes by using a large number of data sets including the morphological information extracted for the lesion candidate, the blood inspection information, and the correct answer information indicating whether the ischemic heart disease has developed later with the lesion candidate as the culprit lesion for the training data and performing learning using an algorithm such as a backpropagation method. In the present embodiment, the trained learning model MD7 is stored in the auxiliary storage unit 35.

Note that, in the present embodiment, the information on the onset risk of ischemic heart disease (ID) is output from the learning model MD7. Alternatively, the information on the onset risk may be output only for acute coronary syndrome (ACS), or the information on the onset risk may be output only for acute myocardial infarction (AMI).

In addition, the learning model MD7 may be installed in an external server, and the external server may be accessed via the communication unit 34 to cause the external server to execute the computation by the learning model MD7.

Furthermore, the control unit 31 may derive the time series transition of the onset risk by inputting the values of stress calculated at a plurality of timings to the learning model MD7.

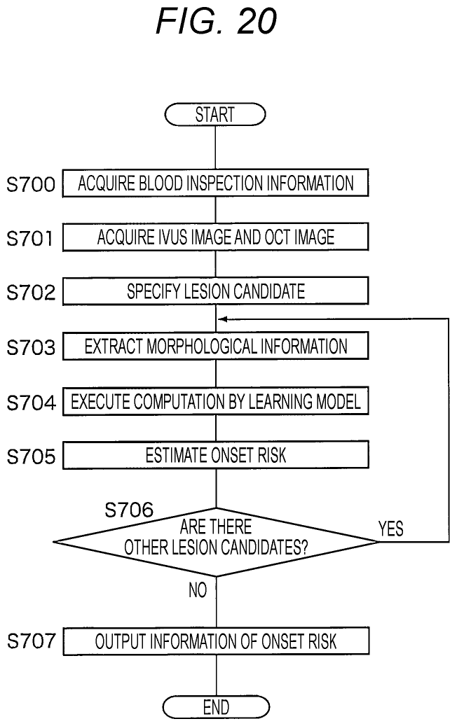

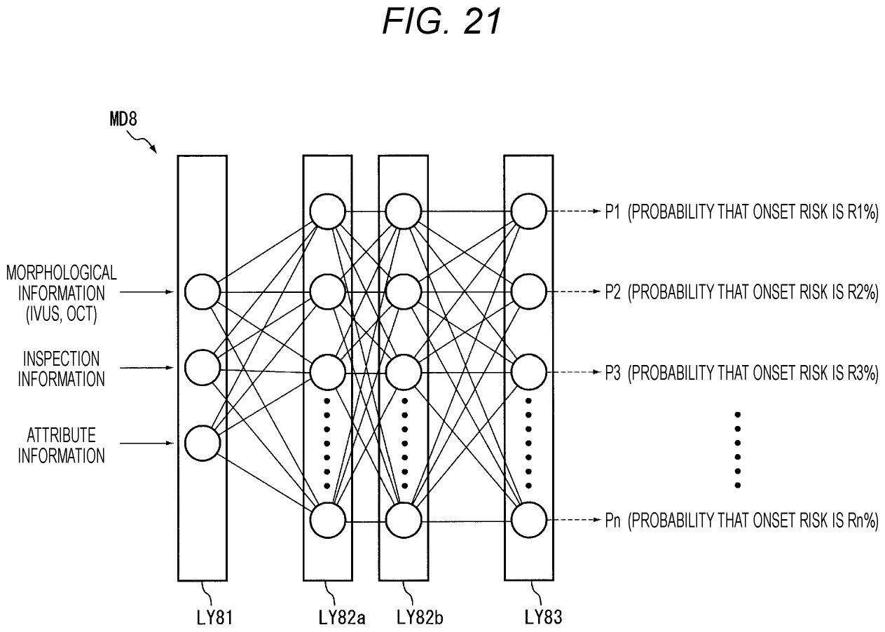

FIG. 20 is a flowchart for explaining a process executed by the image processing apparatus 3 in the seventh embodiment. The control unit 31 of the image processing apparatus 3 executes the onset risk prediction program PG stored in the auxiliary storage unit 35 to perform the following process. The control unit 31 acquires blood inspection information measured in advance (S700). The inspection information may be acquired from external equipment by communication via the communication unit 34, or may be manually input using the input apparatus 5.

The control unit 31 acquires the IVUS image and the OCT image captured by the intravascular inspection apparatus 101 through the input/output unit 33 (S701).

The control unit 31 specifies a lesion candidate for the blood vessel of the patient (S702). For example, the control unit 31 calculates plaque burden from the IVUS image, and determines whether the calculated plaque burden exceeds a preset threshold value (for example, 50%), thereby specifying a lesion candidate. Alternatively, the control unit 31 may specify a lesion candidate using a learning model learned to identify a region such as a calcified region or a thrombus region from an IVUS image, an OCT image, or an angiographic image. In S702, one or a plurality of lesion candidates may be specified.

The control unit 31 extracts morphological information in the specified lesion candidate (S703). The method of extracting morphological information is similar to that of the first embodiment, and feature amounts (i.e., first feature amounts) related to forms such as attenuated plaque (e.g., a lipid core), remodeling index, calcified plaque, neovessels, and plaque volume are extracted from the IVUS image, and feature amounts (i.e., second feature amounts) related to forms such as the thickness of the fibrous cap, neovessels, calcified plaque, lipid plaque, and infiltration of macrophages are extracted from the OCT image.

The control unit 31 inputs the extracted morphological information and the acquired blood inspection information to the learning model MD7 and executes computation by the learning model MD7 (S704). The control unit 31 gives the morphological information and the inspection information to the nodes provided in the input layer LY71 of the learning model MD7, and sequentially executes the computation in the intermediate layer LY72 according to the trained internal parameters (e.g., weight coefficient and bias). The computation result by the learning model MD7 is output from each node of the output layer LY73.

The control unit 31 refers to the information output from the output layer LY73 of the learning model MD7 and estimates the onset risk of ischemic heart disease (S705). For example, since information regarding the probability of the onset risk is output from each node of the output layer LY73, the control unit 31 can estimate the onset risk by selecting a node having the highest probability. The control unit 31 may derive the time series transition of the onset risk by inputting the morphological information extracted at a plurality of timings and the inspection information acquired in advance to the learning model MD7 and performing computation.

The control unit 31 determines whether there are other specified lesion candidates (S706). When it is determined that there is another specified lesion candidate (S706: YES), the control unit 31 returns the process to S703.

When it is determined that there are no other specified lesion candidates (S706: NO), the control unit 31 outputs information on the onset risk estimated in S705 (S707). The output method is similar to that of the first embodiment. For example, as illustrated in FIG. 9, a graph indicating the level of the onset risk for each lesion candidate may be generated and displayed on the display apparatus 4, or a graph indicating the time series transition of the onset risk for each lesion candidate as illustrated in FIG. 10 may be generated and displayed on the display apparatus 4. Alternatively, the control unit 31 may notify the external terminal or the external server of the information on the onset risk through the communication unit 34.

As described above, in the seventh embodiment, since the onset risk of ischemic heart disease is estimated based on the morphological information extracted from the lesion candidate and the blood inspection information, it is possible to accurately estimate the onset risk of ischemic heart disease, which is conventionally considered difficult.

### Eighth Embodiment

In an eighth embodiment, a configuration for estimating the onset risk of ischemic heart disease based on morphological information of a lesion candidate, blood inspection information, and attribute information of a patient will be described.

Since the overall configuration of the image diagnosis system 100, the internal configuration of the image processing apparatus 3, and the like are similar to those of the first embodiment, the description thereof will be omitted.

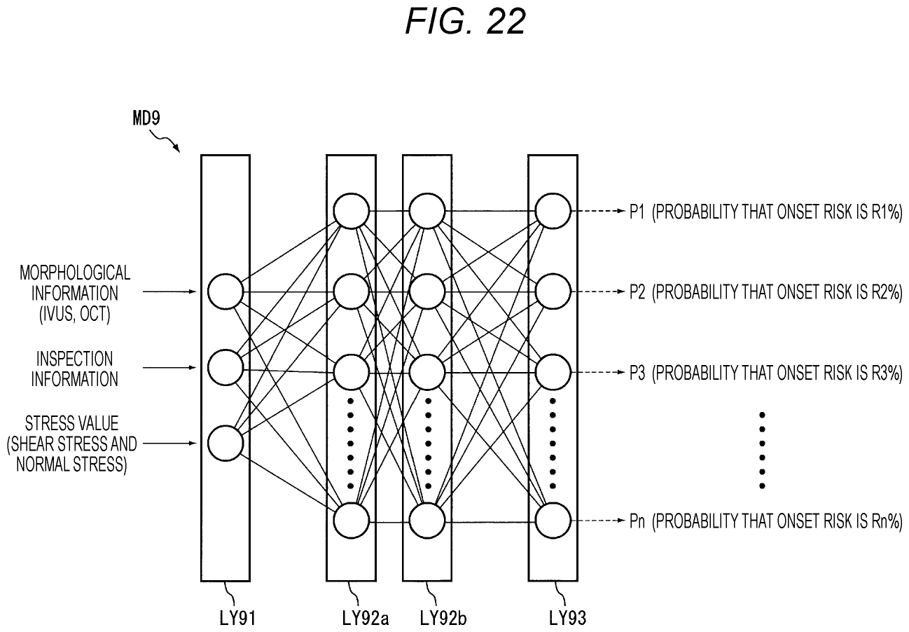

FIG. 21 is a schematic diagram illustrating a computer learning model MD8 in the eighth embodiment. The configuration of the learning model MD8 is similar to that of the first embodiment, and includes an input layer LY81, intermediate layers LY82a and 82b, and an output layer LY83. An example of the learning model MD8 is a DNN. Alternatively, SVM, XGBoost, LightGBM, or the like is used.

The input data in the eighth embodiment is morphological information of a lesion candidate, blood inspection information, and attribute information of a patient. The morphological information of the lesion candidate and the blood inspection information are similar to those in the seventh embodiment and the like. As the attribute information of the patient, information generally confirmed as a background factor of the PCI patient, such as age, sex, weight, and co-morbidity of the patient, is used. The attribute information of the patient is input to the image processing apparatus 3 through the communication unit 34 or the input apparatus 5.

The data provided to each node of the input layer LY81 is provided to the first intermediate layer LY82a. The output is calculated in the intermediate layer LY82a using the activation function including the weight coefficient and the bias, the calculated value is given to the next intermediate layer LY82b, and the output of the output layer LY83 is successively transmitted to the subsequent layers until the output is obtained in the same manner.

The output layer LY83 outputs information related to the onset risk of ischemic heart disease. The output form of the output layer LY83 is any form. For example, n pieces (n is an integer of 1 or more) may be provided in the output layer LY83, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY83 of the learning model MD8 and estimate the highest probability as the onset risk of ischemic heart disease.

Furthermore, the learning model MD8 may be designed so as to predict the presence or absence of onset within a predetermined number of years (for example, within three years), and information of 0 (=no onset) or 1 (=onset) may be output from the output layer LY83. Furthermore, the learning model MD8 may be designed so as to calculate the probability of onset within a predetermined number of years (for example, within three years), and the probability (a real value of 0 to 1) may be output from the output layer LY83. In these cases, the number of nodes provided in the output layer LY83 may be one.

The learning model MD8 is trained according to a predetermined learning algorithm, and internal parameters (e.g., weight coefficient, bias, or the like) are determined. Specifically, it is possible to determine the internal parameters of the learning model MD8 including the weight coefficient and the bias between the nodes by using a large number of data sets including the morphological information extracted for the lesion candidate, the blood inspection information, patient attribute information, and the correct answer information indicating whether the ischemic heart disease has developed later with the lesion candidate as the culprit lesion for the training data and performing learning using an algorithm such as a backpropagation method. In the present embodiment, the trained learning model MD8 is stored in the auxiliary storage unit 35.

In the operation phase after completion of the learning, the control unit 31 of the image processing apparatus 3 inputs the morphological information extracted for the lesion candidate, the blood inspection information, and the attribute information of the patient to the learning model MD8, and executes computation by the learning model MD8. The control unit 31 refers to the information output from the output layer LY83 of the learning model MD8 and estimates the highest probability as the onset risk of ischemic heart disease.

Note that, in the present embodiment, the information on the onset risk of ischemic heart disease (ID) is output from the learning model MD8. Alternatively, the information on the onset risk may be output only for acute coronary syndrome (ACS), or the information on the onset risk may be output only for acute myocardial infarction (AMI).

In addition, the learning model MD8 may be installed in an external server, and the external server may be accessed via the communication unit 34 to cause the external server to execute the computation by the learning model MD8.

Furthermore, the control unit 31 may derive the time series transition of the onset risk by inputting the values of stress calculated at a plurality of timings to the learning model MD8.

### Ninth Embodiment

In a ninth embodiment, a configuration for estimating the onset risk of ischemic heart disease based on morphological information of a lesion candidate, blood inspection information, and a value of stress applied to the lesion candidate will be described.

Since the overall configuration of the image diagnosis system 100, the internal configuration of the image processing apparatus 3, and the like are similar to those of the first embodiment, the description thereof will be omitted.

FIG. 22 is a schematic diagram illustrating a computer learning model MD9 in the ninth embodiment. The configuration of the learning model MD9 is similar to that of the first embodiment, and includes an input layer LY91, intermediate layers LY92a and 92b, and an output layer LY93. An example of the learning model MD9 is a DNN. Alternatively, SVM, XGBoost, LightGBM, or the like is used.

The input data in the ninth embodiment is morphological information of a lesion candidate, blood inspection information, and a value of stress applied to the lesion candidate. The morphological information of the lesion candidate and the blood inspection information are similar to those in the seventh embodiment and the like, and the value of the stress applied to the lesion candidate is calculated by a method similar to that in the third embodiment.

The data provided to each node of the input layer LY91 is provided to the first intermediate layer LY92a. The output is calculated in the intermediate layer LY92a using the activation function including the weight coefficient and the bias, the calculated value is given to the next intermediate layer LY92b, and the output of the output layer LY93 is successively transmitted to the subsequent layers until the output is obtained in the same manner.

The output layer LY93 outputs information related to the onset risk of ischemic heart disease. The output form of the output layer LY93 is any form. For example, n pieces (n is an integer of 1 or more) may be provided in the output layer LY93, and a probability (=P1) that the onset risk is R1% from the first node, a probability (=P2) that the onset risk is R2% from the second node, . . . , and a probability (=Pn) that the onset risk is Rn % from the nth node may be output. The control unit 31 of the image processing apparatus 3 can refer to the information output from the output layer LY93 of the learning model MD9 and estimate the highest probability as the onset risk of ischemic heart disease.

Furthermore, the learning model MD9 may be designed so as to predict the presence or absence of onset within a predetermined number of years (for example, within three years), and information of 0 (=no onset) or 1 (=onset) may be output from the output layer LY93. Furthermore, the learning model MD9 may be designed so as to calculate the probability of onset within a predetermined number of years (for example, within three years), and the probability (a real value of 0 to 1) may be output from the output layer LY93. In these cases, the number of nodes provided in the output layer LY93 may be one.

The learning model MD9 is trained according to a predetermined learning algorithm, and internal parameters (e.g., weight coefficient, bias, or the like) are determined. Specifically, it is possible to determine the internal parameters of the learning model MD9 including the weight coefficient and the bias between the nodes by using a large number of data sets including the morphological information extracted for the lesion candidate, the blood inspection information, a value of stress applied to a lesion, and the correct answer information indicating whether the ischemic heart disease has developed later with the lesion candidate as the culprit lesion for the training data and performing learning using an algorithm such as a backpropagation method. In the present embodiment, the trained learning model MD9 is stored in the auxiliary storage unit 35.

In the operation phase after completion of the learning, the control unit 31 of the image processing apparatus 3 inputs the morphological information extracted for the lesion candidate, the blood inspection information, and the attribute information of the patient to the learning model MD9, and executes computation by the learning model MD9. The control unit 31 refers to the information output from the output layer LY93 of the learning model MD9 and estimates the highest probability as the onset risk of ischemic heart disease.

Note that, in the present embodiment, the information on the onset risk of ischemic heart disease (ID) is output from the learning model MD9. Alternatively, the information on the onset risk may be output only for acute coronary syndrome (ACS), or the information on the onset risk may be output only for acute myocardial infarction (AMI).

In addition, the learning model MD9 may be installed in an external server, and the external server may be accessed via the communication unit 34 to cause the external server to execute the computation by the learning model MD9.

Furthermore, the control unit 31 may derive the time series transition of the onset risk by inputting the values of stress calculated at a plurality of timings to the learning model MD9.

It should be understood that the embodiments disclosed herein are illustrative in all respects and are not restrictive. The technical features described in the examples can be combined with each other. The scope of the present invention is defined not by the meanings described above but by the claims, and is intended to include meanings equivalent to the claims and all modifications within the scope.

What is claimed is:

1. An image diagnosis system comprising:
a catheter insertable into a blood vessel and including:
a first sensor configured to transmit ultrasonic waves and receive the waves reflected by the blood vessel while the catheter is inserted in the blood vessel, and
a second sensor configured to emit light and receive the light reflected by the blood vessel while the catheter is inserted in the blood vessel;
a memory; and
a processor configured to execute a program that is stored in the memory to perform the steps of:
generating an ultrasonic tomographic image of the blood vessel based on the reflected waves received by the first sensor and an optical coherence tomographic image of the blood vessel based on the reflected light received by the second sensor,
specifying a location of a lesion in the blood vessel based on the ultrasonic tomographic image and the optical coherence tomographic image,
generating first feature data related to the lesion from the ultrasonic tomographic image and second feature data related to the lesion from the optical coherence tomographic image,
inputting the first and second feature data into a computer model to generate risk information related to an onset risk of ischemic heart disease, the computer model having been trained with feature data of different lesions and a plurality of answer information corresponding to the different lesions, each answer information indicating whether the ischemic heart disease has developed from a corresponding one of the lesions, and
outputting the risk information related to the onset risk of ischemic heart disease.

2. The image diagnosis system according to claim 1, wherein
the first feature data indicates a feature of at least one of an attenuated plaque, a remodeling index, a calcified plaque, a neovessel, and a plaque volume.

3. The image diagnosis system according to claim 1, wherein
the second feature data indicates a feature of at least one of a thickness of a fibrous cap, a neovessel, a calcified plaque, a lipid plaque, and infiltration of macrophages.

4. The image diagnosis system according to claim 1, wherein
specifying the location includes calculating a plaque burden using the ultrasonic tomographic image and determining that the plaque burden exceeds a threshold.

5. The image diagnosis system according to claim 1, further comprising:
a display, wherein
the steps further include controlling the display to display the risk information.

6. The image diagnosis system according to claim 5, wherein
the steps further include controlling the display to display the ultrasonic tomographic image and the optical coherence tomographic image.

7. The image diagnosis system according to claim 1, wherein
the steps include calculating a value of stress applied to the blood vessel by the lesion, and
the calculated value of stress is further input to the computer model.

8. The image diagnosis system according to claim 1, wherein
the steps include obtaining blood information about a blood inside the blood vessel, and
the obtained blood information is further input to the computer model.

9. The image diagnosis system according to claim 1, further comprising:
an angiography apparatus configured to generate an angiographic image of the blood vessel, wherein
the catheter includes a marker that can be imaged by the angiography apparatus.

10. The image diagnosis system according to claim 9, wherein
the marker is adjacent to the second sensor.

11. An image diagnosis method performed by an image diagnosis system that includes a catheter insertable into a blood vessel and including: a first sensor configured to transmit ultrasonic waves and receive the waves reflected by the blood vessel while the catheter is inserted in the blood vessel, and a second sensor configured to emit light and receive the light reflected by the blood vessel while the catheter is inserted in the blood vessel,
the image diagnosis method comprising:
generating an ultrasonic tomographic image of the blood vessel based on the reflected waves received by the first sensor and an optical coherence tomographic image of the blood vessel based on the reflected light received by the second sensor;
specifying a location of a lesion in the blood vessel based on the ultrasonic tomographic image and the optical coherence tomographic image;
generating first feature data related to the lesion from the ultrasonic tomographic image and second feature data related to the lesion from the optical coherence tomographic image;
inputting the first and second feature data into a computer model to generate risk information related to an onset risk of ischemic heart disease, the computer model having been trained with feature data of different lesions and a plurality of answer information corresponding to the different lesions, each answer information indicating whether the ischemic heart disease has developed from a corresponding one of the lesions; and
outputting the risk information related to the onset risk of ischemic heart disease.

12. The image diagnosis method according to claim 11, wherein
the first feature data indicates a feature of at least one of an attenuated plaque, a remodeling index, a calcified plaque, a neovessel, and a plaque volume.

13. The image diagnosis method according to claim 11, wherein
the second feature data indicates a feature of at least one of a thickness of a fibrous cap, a neovessel, a calcified plaque, a lipid plaque, and infiltration of macrophages.

14. The image diagnosis method according to claim 11, wherein
specifying the location includes calculating a plaque burden using the ultrasonic tomographic image and determining that the plaque burden exceeds a threshold.

15. The image diagnosis method according to claim 11, further comprising:
displaying the risk information.

16. The image diagnosis method according to claim 15, further comprising:
displaying the ultrasonic tomographic image and the optical coherence tomographic image.

17. The image diagnosis method according to claim 11, further comprising:
calculating a value of stress applied to the blood vessel by the lesion, wherein
the calculated value of stress is further input to the computer model.

18. The image diagnosis method according to claim 11, further comprising:
obtaining blood information about a blood inside the blood vessel, wherein
the obtained blood information is further input to the computer model.

19. The image diagnosis method according to claim 11, further comprising:
generating an angiographic image of the blood vessel and a marker of the catheter.

20. A non-transitory computer readable medium storing a program causing a computer to execute an image diagnosis method comprising:
generating an ultrasonic tomographic image of a blood vessel based on reflected waves received by a first sensor of a catheter and an optical coherence tomographic image of the blood vessel based on reflected light received by a second sensor of the catheter;
specifying a location of a lesion in the blood vessel based on the ultrasonic tomographic image and the optical coherence tomographic image;
generating first feature data related to the lesion from the ultrasonic tomographic image and second feature data related to the lesion from the optical coherence tomographic image;
inputting the first and second feature data into a computer model to generate risk information related to an onset risk of ischemic heart disease, the computer model having been trained with feature data of different lesions and a plurality of answer information corresponding to the different lesions, each answer information indicating whether the ischemic heart disease has developed from a corresponding one of the lesions; and
outputting the risk information related to the onset risk of ischemic heart disease.
